(19) United States
(12) Patent Application Publication
(10) Pub. No.: US 2005/0244505 A1
(43) Pub. Date: Nov. 3, 2005

(54) Immunotherapy compositions, method of making and method of use thereof

(76) Inventors: Russell G. Higbee, Orlando, FL (US); Glen N. Barber, Miami, FL (US); Anatoly M. Kachurin, Orlando, FL (US); Olga M. Kachurina, Orlando, FL (US); Heather Gappa-Fahlekamp, Oviedo, FL (US); William L. Warren, Orlando, FL (US); Siddharth Balachandran, Miami, FL (US); Emmanuel Thomas, Miami, FL (US); Robert Parkhill, Orlando, FL (US)

Correspondence Address: Steven B. Kelber, Esquire;MERCHANT & GOULD, 901 Fifteenth Street, N.W., Suite 850, Washington, DC, 20005, US

(21) Appl. No.: 11/008,936

(22) Filed: Dec. 13, 2004

Related U.S. Application Data
(60) Provisional application No. 60/528,613, filed on Dec. 11, 2003.
(60) Provisional application No. 60/605,554, filed on Aug. 31, 2004.

(51) Int. Cl. A61K 48/00; C12P 21/06; C12N 15/87; A61K 9/14

(52) U.S. Cl. 424/489; 514/44; 435/459

(57) ABSTRACT

The present invention directs to compositions and methods for modulating immune system. One aspect of the present invention relates to a composition comprising FADD-dependent signaling pathway modulators. Another aspect of the present invention relates to biodegradable microparticles, such as a chitosan microparticle, or PLGA/PEI microparticle, designed to deliver nucleic acids and/or proteins, such as FADD-dependent signaling pathway modulators, to boost different pathways of an immune response. Another aspect of the present invention relates to the method of making biodegradable microparticles. The further aspect of the present invention relates to the use of the chitosan and other polycationic microparticles to deliver FADD-dependent signaling pathway modulators to modulate immune system for the prevention and/or treatment infectious diseases and cancers.

This application claims priority from U.S. Provisional Application Ser. No. 60/528,613, filed Dec. 11, 2003 and U.S. Provisional Application Ser. No. 60/605,554, filed Aug. 31, 2004, respectively. The entirety of both provisional applications is incorporated herein by reference.

### BACKGROUND OF THE INVENTION

Field of the Invention

The present invention relates to the field of immunotherapy. More particularly, it relates to compositions capable of activating either or both the endogenous fas-associated death domain molecule (FADD)-RIP1 dependent signaling pathway and the exogenous Toll-like receptor (TLR)-dependent pathway and methods to more effectively couple innate adaptive immune responses. The compositions are particularly useful in modulating innate immune responses against viral, fungal, and bacterial pathogens, as well as in treating cancer.

### BACKGROUND OF THE TECHNOLOGY

A host exposes to microbial pathogens such as viruses, bacteria, and fungi that triggers the activation of innate immune responses that galvanize early host defense mechanisms as well as invigorate adaptive immune responses involving cytotoxic T cell activity and antibody production [Medzhitov, et al., Semin. Immunol., 10:351-353, (1998)]. The recognition of pathogenic microbes and the triggering of the innate immune cascade has become the subject of intense research over the past few years.

Particular attention has recently focused on the role of the Toll-like receptors (TLRs), which have emerged as key surface molecules responsible for recognizing conserved components of pathogenic microorganisms (referred to as pathogen-associated molecular patterns—PAMPs), such as lipopolysaccharide and CpG DNA (FIG. 1) [Medzhitov, et al., Semin. Immunol., 10:351-353, (1998)]. The TLRs were first identified in Drosophila (the fruit fly) and have been demonstrated as playing an important role in fly development as well as in host defense against fungi and gram-positive bacteria [Imler, et al., Curr. Top. Microbiol. Immunol., 270:53-79, (2002)].

Engagement of a TLR transmits a signal to the cell's nucleus, inducing the cell to begin producing certain proteins such as cytokines, alerting other components of host defenses. In mammalian cells, there appear to be at least ten TLR members, each of which respond to different stimuli including extracellular lipopolysaccharide (LPS) and dsRNA [Takeda, et al., Ann. Rev. Immunol., 21:335-376, 2003]. Following ligand binding, signaling pathways are initiated through homophilic interactions triggered by a Toll/interleukin (IL)-1 receptor (TIR) domain present in the cytosolic region of all TLRs [Akira, Jour. Biol. Chem., 278:38105-38108, 2003]. Many TLRs, including TLR-2, -4, and -5, use a common adaptor protein referred to as MYD88, which contains a TIR domain as well as a death domain (DD). Other adaptor molecules that function similarly to MYD88 (though lack a DD) referred to as TRIF/TICAM, TRAM, and TIRAP/Mal have now been isolated and similarly function in the modulation of TLR activity [Horng, et al., Nat. Immunol., 2:835-841, (2001); Oshiumi, et al., Nat. Immunol., 4:161-167, (2003); Yamamoto, et al., Science, 301:640-643, (2003); Yamamoto, et al., Natl. Immunol., 4:1144-1150, (2003)]. The resident DD of MYD88 probably facilitates interaction with members of the IL-1 receptor-associated kinase (IRAK) family such as IRAK-1 and -4 which are DD-containing serine-threonine kinases involved in the phosphorylation and activation of TRAF-6 [Cao, et al., Science, 271:1128-1131, (1996); Ishida, etal., J. Biol. Chem., 271:28745-28748, (1996); Muzio, et al., Science, 278:1612-1615, (1997); Suzuki, et al., Nature, 416:750-756, (2002)].

All TLRs trigger common signaling pathways that culminate in the activation of the transcription factors NF-κB as well as the mitogen-activated protein kinases (MAPKs), extracellular signal-regulated kinase (ERK), p38, and c-Jun N-terminal kinase (JNK) [Akira, J. Biol. Chem., 278:38105-38108, (2003)]. In addition, stimulation of TLR-3 or -4 can activate the transcription factor interferon regulatory factor (IRF)-3, perhaps through TRIF-mediated activation of the noncanonical IκB kinase homologues, IκB kinase-ε (IKKε), and TANK-binding kinase-1 (TBK1), although the exact mechanisms remain to be clarified [Doyle, et al., Immunity, 17:251-263, (2002); Fitzgerald, et al., Nat. Immunol., 4:491-496, (2003)].

Activation of the NF-κB, ERK/JNK, and IRF-3 responsive signaling cascades culminates in the transcriptional stimulation of numerous genes that regulate the innate and adaptive immune responses including the inflammatory response.

Activation of primary innate immune response genes such as IFN-β induces not only anti-viral genes, but also molecules that facilitate innate immune responses involving NK cells, the maturation of DCs as well as upregulation of chemokines and molecules such as MHC that facilitate T-cell responses. IFN has also been shown to be critically important for the production of antibody responses. Thus, understanding and potentially regulating the innate immune responses affords the opportunity to develop novel therapeutic and vaccination methods and compositions targeting disease for both innate and adaptive immune responses.

An important aspect of immunotherapy is the development of an effective drug/antigen delivery system. Particle carriers have been devised to deliver drugs, antigens and other signal molecules to cells [Aideh, et al., J. Microencapsul., 14:567-576 (1997); Akbuga, et al., Microencapsul., 13:161-167 (1996); Akbuga, et al., Int. J. (1994); Aral, et al., STP Pharm. Sci., 10:83-88 (2000)]. Requirements of these delivery carriers differ depending on application. For example, carriers of chemokines need to provide stable gradients of the loaded molecules for an extended period of time (usually days) and the particles need to be relatively large (200-700 μm) to avoid being phagocytosed.

On the other hand, immunization is stronger when antigens are carried by smaller particles that not only interact with cells via their surface, but can also be engulfed by dendritic cells, macrophages or other antigen presenting cells (APCs). Phagocytosis is optimal for the particles smaller than 10 μm, which stipulates sizes for antigen carriers.

Chitosan is a natural product derived from chitin. It is chemically similar to cellulose, which is the major composition of plant fiber, and possesses many properties as fiber. Chitosan has been shown to exhibit high adhesion to mucosa and good biodegradability, as well as ability to enhance penetration of large molecules across mucosal surfaces [Illum, et al., Pharm. Res., 9:1326-1331 (1992)]. Chitosan nanoparticles have been demonstrated to be very efficient in improving the nasal absorption of insulin, as well as in the local and systemic immune responses to tetanus toxoid [Vila, et al.,J. Controlled Release, 17;78(1-3): 15-24 (2002)]. Similar boost of immune system was demonstrated in mucosal vaccination with chitosan microparticles against diphtheria [Inez, et al., Vaccine, 21:1400-1408 (2003)]: protective systemic and local immune response against DR after oral vaccination and significant enhancement of IgG production after nasal administration. Recently, chitosan has shown promise as a carrier for delivery drugs to the colon [Zhang, et al., Biomaterials, 23:2761-2766 (2002)].

### SUMMARY OF THE INVENTION

One aspect of the present invention relates to a composition for modulating innate immune system in a mammal. The composition comprises: a microparticle comprising a polycationic polymer; a modulator of FADD-dependent pathway; and a modulator of TLR pathway, wherein said modulator of FADD-dependent pathway and said modulator of TLR pathway are associated with said microparticle, and wherein said microparticle is capable of being phagocytosed by an antigen presenting cell.

In one embodiment, the modulator of FADD-dependent pathway is selected from the group consisting of double-stranded RNA (dsRNA), poly(IC), a component of the FADD-dependent pathway, a DNA plasmid encoding a component of the FADD-dependent pathway, a bacterium, and a fungus.

In another embodiment, the modulator of TLR pathway is selected from the group consisting of dsRNA, poly (IC), a synthetic mimetic of viral dsRNA, and a ligand for TLR, a bacterium, and a fungus.

In another embodiment, the microparticle is further coated with a targeting molecule that binds specifically to an antigen presenting cell.

Another aspect of the present invention relates to a composition for modulating immune system in a host, comprising phagocytosable chitosan microparticles loaded with a nucleic acid and a protein.

Yet another aspect of the present invention relates to a method for treating viral, bacterial, fungal infection and cancer in a subject, comprising administering to said subject an effective amount of the composition described above.

Yet another aspect of the present invention relates to a method for preparing a multifinctional microparticle for immune modulation. The method comprises the steps of fabricating chitosan microparticles by precipitation, gelation and spray; and incubating the chitosan microparticles in a solution comprising a nucleic acid, a protein, or both.

Another aspect of the invention relates to creating particles with multiple/multifunctional agents that can activate both innate and adaptive immune responses.

Yet another aspect of the present invention relates to a method for identifying anti-viral genes relating to FADD signaling pathway. The method comprises the steps of treating FADD-deficient cells and corresponding wild-type cells with poly (IC); isolating RNAs from poly (IC)-treated FADD-deficient cells and poly (IC)-treated wild-type cells; hybridizing the isolated RNAs to a gene array; and identifying genes that are differentially expressed in poly (IC)-treated FADD-deficient cells comparing to poly (IC)-treated wild-type cells.

### BRIEF DESCRIPTION OF THE FIGURES

FIG. 1 illustrates the detection of PAMPs by a host cell through TLRs.

FIG. 2 illustrates the antiviral mechanism of interferons.

FIG. 3 is a schematic of the TNF-α pathway.

FIG. 4 is a schematic of the pathways of antigen processing and delivery to Major Histocompatibility Complex (MHC) molecules.

FIG. 5 is a schematic of Poly(IC) treatment protocol.

FIG. 6 is a schematic of the proposed method of enhancing innate immunity by activating two viral signaling pathways, exogenous TLR-3 and endogenous FADD-dependent pathways, to produce INF.

FIG. 7 is a structural formulation of chitosan.

FIG. 8 is a microscopic picture showing polystyrene beads phagocytosed by a monocyte-derived human dendritic cell.

FIG. 9 is a structural formula of branched PEI.

FIG. 10 is the artificial virus-like particles consisting of (1) yeast dsRNA, (2) spermidine-polyglucin-glutathione conjugate, and (3) hybrid protein TBI-GST.

FIGS. 11a-11f are experimental data showing that FADD, but not caspase-8, is required for prevention of VSV replication in MEFs even after IFN pretreatment. FIG. 11a shows that FADD-deficient MEFs are susceptible to VSV-induced CPE despite IFN pretreatment and photomicrographs were taken 48 hours post-infection. FIG. 11b shows that FADD-deficient MEFs are not protected from VSV-triggered cell death by IFN pretreatment. Cell viability was determined at the indicated times post-infection by Trypan Blue exclusion analysis. FIG. 11c shows that IFN pretreatment delays, but does not prevent, VSV replication in FADD −/− EFs. FIG. 11d shows that caspase-8 deficiency does not predispose MEFs to increased susceptibility to VSV induced CPE. FIG. 11e shows that caspase-8+/+ and −/− MEFs are equally well-protected from VSV-induced cell death by IFN pretreatment. FIG. 11f shows that IFN pretreatment efficiently inhibits VSV replication in both caspase-8+/+ and −/− EFs.

FIGS. 12a-12d are experimental data illustrating that absence of FADD sensitizes cells to the infection by encephalomyocarditis virus (EMCV) and influenza virus (FLU) infection. FIG. 12a shows that FADD is required to protect against EMCV-induced CPE. Cells were photographed (Mag. 200×) 24 hours post infection. FIG. 12b shows that cells infected as in (a) were analyzed for cell viability by Trypan Blue exclusion. FIG. 12c shows that FADD is required to protect against EMCV-induced CPE. Cells were photographed (Mag. 200×) 24 hours post infection. FIG. 12d shows that cells infected as in (c) were analyzed for cell viability by Trypan Blue exclusion.

FIGS. 13a-13f are experimental data illustrating that IFN signaling is not disrupted in FADD−/− MEFs. FIG. 13a shows normal STAT1 phosphorylation in the absence of FADD. FIG. 13b shows that nuclear translocation of STAT1 following IFN treatment occurs normally in the absence of FADD. FIG. 13c shows that FADD is not required for IFN-triggered gene induction. FIG. 13d shows IFN-responsive promoters function normally in the absence of FADD. FIG. 13e shows that exogenous IFN-β can protect FADD−/− MEFs from VSV-induced CPE when added after infection. Cells were photographed 48 hours post-infection. FIG. 13f shows exogenous IFN-β can protect FADD−/− MEFs from VSV replication and consequent cell death when added after infection.

FIGS. 14a and 14b are experimental data illustrating that De Novo synthesis of IFN-β is required to afford continued protection of wild type MEFs following VSV infection despite IFN-α/β pretreatment. FIG. 14a shows that FADD+/− cells are susceptible to VSV in the presence of neutralizing anti-IFN-β antiserum despite IFN-α/β pretreatment. Photographs were taken 48 hours post infection (mag. 200×). FIG. 14b shows that FADD+/− cells treated as in (a) were examined for VSV progeny yield or cell viability by Trypan Blue exclusion.

FIGS. 15a-15g are experimental data illustrating that defective IFN-β gene induction by intracellular dsRNA in the absence of FADD. FIG. 15a shows that transfected dsRNA-mediated activation of the IFN-β promoter is defective in FADD−/− MEFs. FIG. 15b shows that dsRNA-induced production of IFN-α is defective in the absence of FADD. FIG. 15c shows that reconstitution of murine (M) FADD into FADD−/− MEFs can partially rescue dsRNA signaling FIG. 15d shows that caspase-8 is not required for intracellular dsRNA signaling. FIG. 15e shows that PKR is not required for intracellular dsRNA signaling. PKR+/+ and PKR−/− MEFs were transfected with IFN-β-Luc. FIG. 15f shows that RNAi-mediated knockdown of FADD, but not PKR or TLR3 abolishes intracellular dsRNA signaling. FIG. 15g shows that overexpression of TLR3 confers responsiveness to extracellular, but not intracellular dsRNA.

FIGS. 16a-16e are experimental data showing that TRL3 signaling does not require FADD. FIG. 16a shows that TLR3 and other TLR signaling components induce IFN-β normally in FADD−/− MEFs. FIG. 16b shows that TRAF6 deficiency does not predispose MEFs to VSV infection in the presence of IFN. Photomicrographs were taken 48 hours post-infection. FIGS. 16c and 16d show that TRAF6−/− EFs are protected from VSV-triggered cell death by IFN pretreatment. Cell viability was determined by Trypan Blue exclusion analysis 48 hours post-infection. FIG. 16e shows that IFN Pretreatment protects TRAF6−/− MEFs from VSV.

FIGS. 17a-17f are experimental data showing that RIP deficiency mimics FADD ablation. FIG. 17a shows that RIP-deficient EFs are very susceptible to VSV-induced CPE despite IFN pretreatment. FIG. 17b shows that RIP-deficient EFs are not protected from VSV-triggered cell death by IFN pretreatment. FIG. 17c shows that IFN pretreatment cannot efficiently inhibit virus replication in the absence of RIP. FIGS. 17d and 17e show the defective intracellular dsRNA signaling in the absence of RIP. FIG. 17f shows that RIP is not required for TLR3 signaling.

FIGS. 18a-18j are experimental data illustrating that the antiviral pathway incorporating FADD signals via TBK-1/IKK-δ and IRF-3. FIG. 18a shows infection of wild-type or IKK-α-, IKK-β-, IKK-γ- and IKK-δ-deficient MEFs with VSV (MOI ¼ 10) with or without IFN-α/β (100 Uml21) pre-treatment. FIG. 18b is the DNA microarray analysis of a selected set of antiviral genes. FIG. 18c shows IFN-β production after transfection with poly(I:C), or treatment with poly(I:C) alone. FIG. 18d shows IFN-α production after transfection with the indicated amounts of poly(I:C), or treatment with poly(I:C) alone. FIG. 18e is the localization of IRF-3 after transfection of poly(I:C) for 1 h in FADD+/− and FADD−/− cells. FIG. 18f is the defective IRF-3- responsive promoter activation in FADD−/− MEFs. FIG. 18g is the infection of Irf3+/+ and Irf3−/− MEFs with VSV (MOI ¼ 10) with or without IFN-α/β (100 Uml21) or IFN-γ (0.5 ng ml21) pre-treatment. FIG. 18h shows IFN-β production after transfection with poly(I:C), or treatment with poly(I:C) alone. FIG. 18i shows IFN-α production after transfection with poly(I:C), or treatment with poly(I:C) alone. FIG. 18j is the DNA microarray analysis for a selected set of antiviral genes. Error bars indicate mean ±s.d.

FIGS. 19a-19c are experimental data illustrating that FADD−/− Cells are susceptible to infection by gram-positive and gram-negative intracellular bacteria. FIG. 19a shows that FADD−/− cells are very susceptible to CPE induced by intracellular Listeria infection. FIG. 19b shows that FADD−/− cells are susceptible to cell death induced by intracellular Listeria infection. FIG. 19c shows that FADD−/− cells are very susceptible to CPE induced by intracellular Salmonella infection.

FIG. 20 is a Modified Electrospray device with turbulent receiver.

FIG. 21 is an ESEM image of Chitosan Microparticles prepared by Modified Electrospray with turbulent agitation.

FIG. 22 is a structural formula for polyinosinic-polycytidylic acid, poly(IC).

FIG. 23 is a structural formula for Ethidium Homodimer.

FIG. 24 shows a calibration curve for measuring poly(IC) by fluorescence of intercalated Ethidium Homodimer.

FIGS. 25a and 25b show a comparison of measuring loose and bound poly(IC) using intercalating Ethidium Homodimer intercalator.

(A) Measuring free poly(IC) in solution;

(B) Measuring bound poly(IC) in micro-particles. 1- Illuminator, 2-Detector, 3- Filters, 4- Plate well.

FIG. 26 shows time dependent fluorescent of the chitosan particles loaded with poly(IC) upon their interaction with Ethidium Homodimer.

FIG. 27 shows time release of poly(IC) from the chitosan microparticles.

FIGS. 28a and 28b show purple complexes of monovalent copper with proteins and Bicinchoninic Acid.

A is Biuret complex with peptide nitrogens.

B is chelate complex with Bicinchoninic Acid.

FIG. 29 shows a calibration curve for the Bicinchoninic Acid assay of Ovalbumin.

FIG. 30 represents time release of Ovalbumin from the chitosan microparticles.

FIGS. 31a and 31b are the SEM images of freeze dried Protasan/poly(IC) particles.

A is supra-micron size particles, X100. The bar shows 200 μm.

B is sub micron size particles, X5000. The bar shows 5 μm.

FIGS. 32a-32c are the sorption of poly(IC) by supra-micron protasan particles at different pH.

A shows the optical spectra of poly(IC) decreasing as a result of sorption.

B shows pellets of the particles after sorption of poly(IC).

C shows sorption capacity of the particles at different pH.

FIGS. 33a and 33b are the sorption properties of PLGA/PEI particles.

A shows the sorption of poly(IC) for the particles obtained by different methods.

B shows sorption of poly(IC) at different pH.

FIGS. 34a and 34b illustrate PLGA/PEI/poly(IC) particles obtained via Electrospray over dry stainless steel electrode with subsequent solubilization.

A is SEM X5000, after solubilization; and

B shows sorption capacity: affected by solubilization at high or low ionic strength.

FIGS. 35a and 35b show the particles of PLGA/PEI/poly(IC).

A is the SEM image X5000; and

B is the fluorescent micrograph of diluted water suspension, X200.

FIG. 36 illustrates the induction of IFN β, and IFN α in DC1 and DC2 subsets of human dendritic cells by PLGA/PEI particles with poly(IC).

FIGS. 37a and 37b show the extracellular TLR 3 induction via microparticles with poly (IC).

FIG. 38 illustrates that DC2 subsets in peripheral human blood samples were exposed to PLGA/PEI or Protosan particles (with or without amalgamated dsRNA) and monitored for IFN α expression after 3-6 hours of exposure to the particles

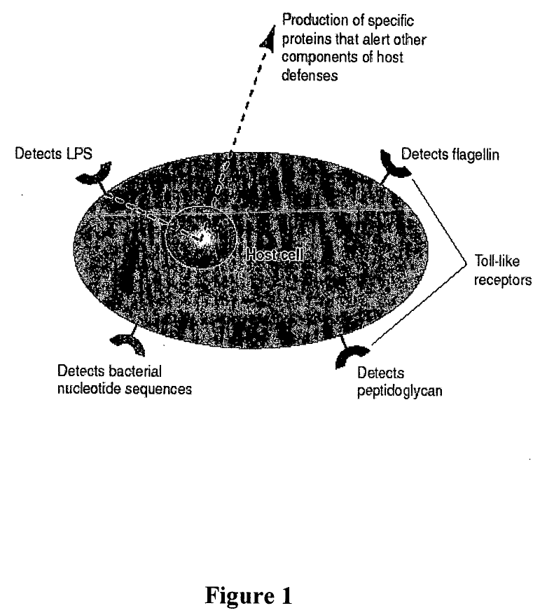

### DETAILED DESCRIPTION OF THE INVENTION

The present invention provides methods and compositions for modulating innate immune responses to antigens. The composition contains an activator for the fas-associated death domain molecule (FADD)/RIP dependent pathway. The signaling pathway incorporating FADD was found to be Toll-Like-Receptor (TLR)-independent and therefore, FADD plays an essential role in innate immunity to viral infection by functioning in the recognition of intracellular dsRNA species, which is critical for the induction of key antiviral responses, including the production of Type I IFN, and that FADD is also involved in the recognition of other pathogens such as bacteria and fungi. As a consequence, the FADD-related pathway is almost certainly a key target for disruption by pathogens and may play a significant role in various diseases including infectious diseases and cancer.

In order to provide a clear and consistent understanding of the specification and claims, including the scope given to such claims, the following definitions are provided:

An “antigen presenting cell” as used hereinafter, refers to a heterogeneous group of immunocompetent cells that mediate the cellular immune response by processing and presenting antigens to the T-cell receptor. Traditional antigen-presenting cells include, but not limited to macrophages, dendritic cells, langerhans cells, and B lymphocytes. Follicular dendritic cells are also considered to be antigen-presenting cells.

The “innate immune response” is the way the body recognizes and defends itself against microorganisms, viruses, and substances recognized as foreign and potentially harmful to the body. The innate immune response functions as a first line of defense against a wide range of infectious and toxic agents. Historically, this response has been attributed to cells with phagocytic activity, such as macrophages and polymorphonuclear cells, and/or potent cytotoxic activity, such as natural killer cells (NK cells), mast cells and eosinophils. The activity of these different cell populations is aided and abetted by a number of different soluble molecules collectively known as acute phase proteins, such as the interferons, specific components of the complement cascade and cytokines, that serve to enhance phagocytic and cytotoxic activity, as well as lead to the accumulation of these cells at sites of tissue injury. If these first lines of defense are breached, then activation of the adaptive immune response ensues, leading to the formation of a specific immune response that may display anyone of a number of different characteristics. The generation of this acquired immune response is an exclusive property of lymphocytes.

In comparison to innate immunity, adaptive immunity develops when the body is exposed to various antigens and builds a defense that is specific to that antigen.

An “immune response” as used hereinafter, refers to an antigen is the development in a mammalian subject of a humoral and/or a cellular immune response to the antigen of interest. A “cellular immune response” is one mediated by T lymphocytes and/or other white blood cells. One important aspect of cellular immunity involves an antigen-specific response by cytotoxic T lymphocytes (“CTL”s). CTLs have specificity for peptide antigens that are presented in association with proteins encoded by the major histocompatibility complex (MHC) and expressed on the surfaces of cells. CTLs help induce and promote the destruction of intracellular microbes, or the lysis of cells infected with such microbes.

The term “antigen” as used herein, refers to any agent (e.g., any substance, compound, molecule [including macromolecules], or other moiety), that is recognized by an antibody, while the term “immunogen” refers to any agent (e.g., any substance, compound, molecule [including macromolecules], or other moiety) that can elicit an immunological response in an individual. These terms may be used to refer to an individual macromolecule or to a homogeneous or heterogeneous population of antigenic macromolecules. It is intended that the term encompasses protein molecules or at least one portion of a protein molecule, which contains one or more epitopes. In many cases, antigens are also immunogenes, thus the term “antigen” is often used interchangeably with the term “immunogen.” The substance may then be used as an antigen in an assay to detect the presence of appropriate antibodies in the serum of the immunized animal.

A “tumor-specific antigen(s)” refers to antigens that are present only in a tumor cell at the time of tumor development in a mammal. For example, a melanoma-specific antigen is an antigen that is expressed only in melanoma cells but not in normal melanocytes.

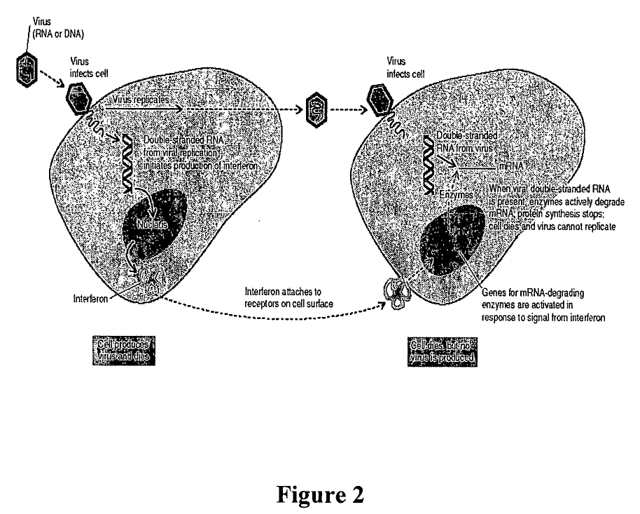

As shown in FIG. 2, a major consequence of viral infection, an event that generates considerable dsRNA species, includes the activation of primary innate immune response genes such as IFN-β. The production of IFN-β induces not only anti-viral genes, but also molecules that facilitate immune responses involving NK cells, the maturation of DCs as well as upregulation of chemokines and molecules such as MHC that facilitate T-cell responses.

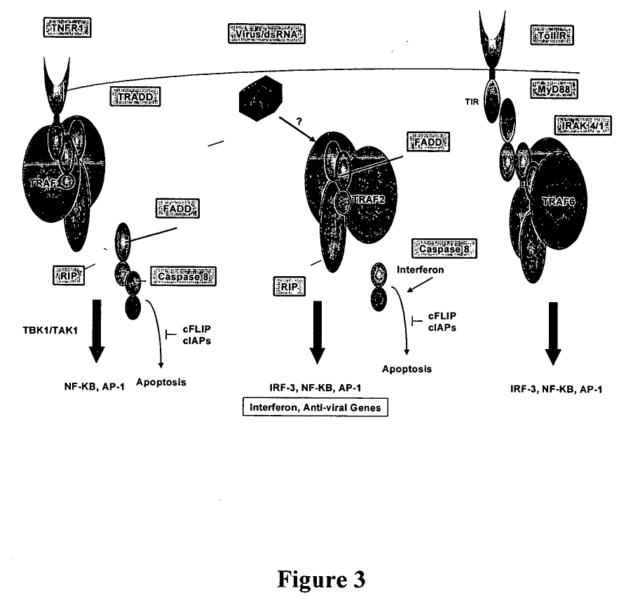

As shown in FIG. 3, intracellular and extracellular dsRNA utilize divergent signaling pathways to induce IFN-β. In particular, intracellular dsRNA species generated as a consequence of virus replication are recognized through a TLR-independent, FADD-related pathway. Briefly, the viral dsRNAs are recognized by an intracellular receptor molecule, which recruits FADD and RIP1 into an ‘innateosome’ complex to activate the NF-κB, ERK/JNK, and IRF-3 pathway. Activation of the NF-κB, ERK/JNK, and IRF-3 responsive signaling cascades leads to the expression of numerous genes that regulate the innate and adaptive immune responses including the inflammatory response. On the other hand, the extracellular PAMPs, including dsRNA and LPS, are recognized through a TLR-related pathway that also leads to the activation of the NF-κB, ERK/JNK, and IRF-3 responsive signaling cascades. In addition to viral infections, both the FADD-dependent and TLR-dependent pathways are also involved in the recognition of other pathogens such as bacteria and fungi (see e.g., Imler et al. Curr. Top. Microbiol. Immunol., 270:53-79, (2002) and Example 6).

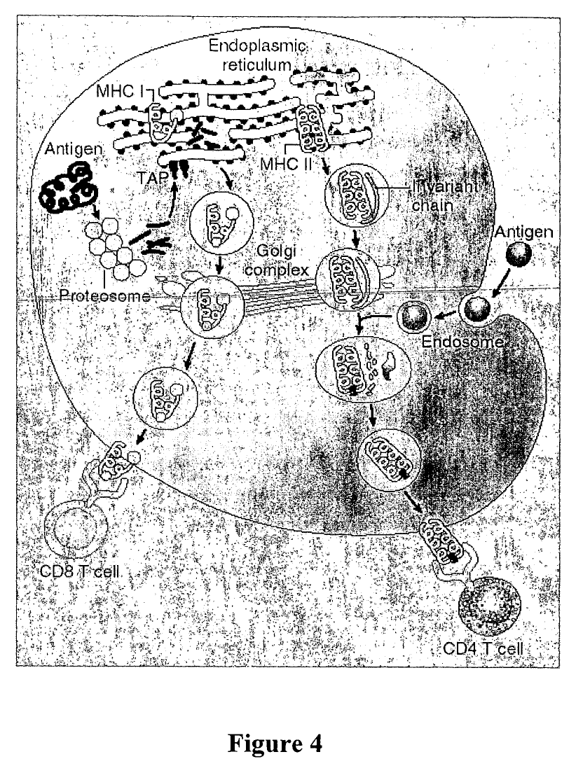

Another key issue in immune activation is the effective delivery of protein antigens by the MHC molecules. The pathways of antigen processing and delivery to MHC molecules as shown in FIG. 4, cytosolic proteins are degraded by the proteosome to generate peptide fragments that are transported into the endoplasmic reticulum by specialized peptide transporters (TAP). After peptides are bound to MHC class I molecules, MHC/peptide complexes are released from the endoplasmic reticulum to travel to the cell surface by the Golgi apparatus. MHC class I/peptide complexes are ligands for T-cell receptors (TCRs) of CD8 T cells. Extracellular foreign antigens are taken into intracellular vesicles, endosomes. As the pH in the endosomes gradually decreases, proteases are activated that digest antigens into peptide fragments. After fusing with vesicles that contain MHC class II molecules, antigenic peptides are placed into the antigen-binding groove. Loaded MHC class II/peptide complexes are transported to the cell surface, where they are recognized by the TCRs of CD4 T cells. Further, as shown in FIG. 4, extracellular or exogenous antigens are phagocytozed by DCs which then localize these antigens to the lysosomal compartment where proteolytic enzymes digest and process the antigen. The antigen is then moved to the cellular surface on class II MHC molecules and never is in the cytosol of the DC. In contrast, soluble proteins present in the cytosol of the DC are continuously degraded by proteasomes. These antigenic molecules are combined with class I MHC in the endoplasmic reticulum which move them to the cell surface via vesicles.

Recently, the strict dichotomy between MHC I and MHC II pathways was challenged by several studies that have shown that peptides generated from exogenous proteins can gain access to the cytosol and therefore be presented on class I MHC molecules [Roake, et al., J. Exp. Med., 181:2237-2247, 1995; Cumbertach, et al., Immunology, 75:257, 1992; Paglia, et al., J. Exp. Med., 178:1893-1901, 1993; Porgador, et al., J. Exp. Med., 182:255-260, 1995; Celluzzi, et al., J. Exp. Med., 183:283-287, 1996; Zitvogel, et al., J. Exp. Med., 183:87-97, 1996; Bender, et al., J. Exp. Med, 182:1663-1671, 1995]. It has been discovered that antigen delivered in a particulate form, either absorbed to solid polymer microspheres [Raychaudhuiri, et al., Nat. Biotechnol. 16:1025-1031, 1998], encapsulated in microspheres [Maloy, et al., IMMUNOLOGY, 81:661-667, 1994], or aggregated in the form of immunocomplexes with antibody [Rodriguez, et al,. Nat. Cell Biol., 1:362-368, 1999], triggers an efficient “cross-presentation” pathway that allows the antigen to be loaded on class I MHC.

Based on this understanding, one aspect of present invention provides compositions for modulating innate immune responses that are capable of cross-signaling both the intracellular and extracellular pathways. In addition, the compositions may trigger the “cross-presentation” pathway that allows the antigen to be loaded on class I MHC and allows the development of an immune reaction against viral or malignant tumor antigens before the viral infection or tumor formation takes place.

In one embodiment, the composition contains a first modulator for the intracellular FADD-dependent signaling pathway and a second modulator for extracellular TLR-independent signaling pathway. The modulators are loaded onto a chitosan-based microparticle that can be phagocytozed by a professional APC such as a DC. As used herein, the term “loaded” refers to the association of the activators to the microparticle, either by encapsulation or by surface attachment.

Examples of modulators of FADD-dependent signaling pathway include, but are not limited to, dsRNA, poly (IC), synthetic mimetic of viral dsRNA, components of FADD-dependent pathway such as FADD and RIP1, DNA encoding a component of FADD pathway, as well as bacteria, fungi, and other antigens that are known to activate or suppress FADD-dependent pathway.

Examples of modulators of TLR-dependent signaling pathway include, but are not limited to, TLR ligands such as dsRNA, poly (IC), synthetic mimetic of viral dsRNA, and LPS; components of TLR-dependent pathway such as MYD88, TRIF/TICAM, TRAM and TIRAP/Mal, as well as bacteria, fungi, and other antigens that are known to activate or suppress TLR-dependent pathway.

It should be noted that a modulator of the FADD-dependent pathway may also function as a modulator of the TLR-dependent pathway. Therefore, the first modulator and the second modulator in the composition of the present invention can be the same molecule. For example, a dsRNA molecule may activate both the FADD-dependent pathway and the TLR-dependent pathway. If the dsRNA encodes a suppressor for FADD-dependent pathway, the same molecule may activate the TLR-dependent pathway while suppressing the FADD-dependent pathway. Vice versa, if the dsRNA encodes a suppressor for TLR-dependent pathway, the same molecule may activate the FADD-dependent pathway while suppressing TLR-dependent pathway.

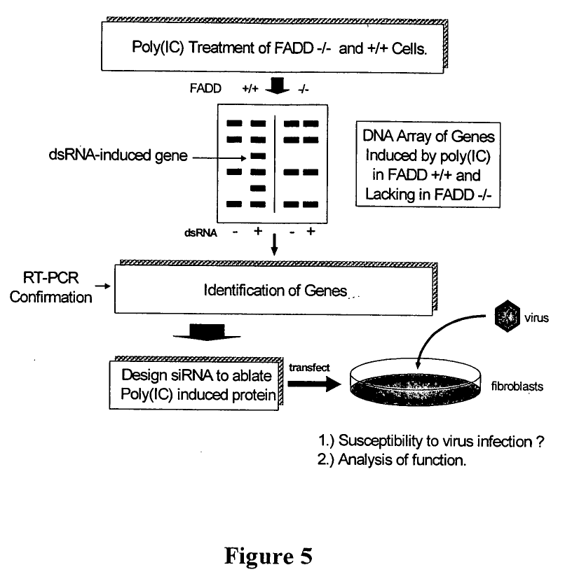

The modulator of the FADD-dependent pathway may also be a gene product that is induced or suppressed by viral, bacterial, or fungal infection. In this regard, the present invention also provides methods for identifying antiviral, anti-bacterial, and anti-fungal genes induced through FADD signaling pathway using FADD−/− and FADD+/+ cells. FIG. 5 depicts one embodiment for identifying antiviral gene induced through FADD signaling pathway. Briefly, FADD−/− and FADD+/+ cells are treated with poly (IC). RNA isolated from the treated cells is hybridized to a DNA array of genes to determine dsRNA-induced genes. The expression levels of the dsRNA-induced genes are further confirmed by quantitative RT-PCR.

In another embodiment, RNA interference (RNAi) is developed to inhibit the expression of dsRNA-induced genes and the susceptibility to viral infection in the RNAi-treated cells is examined. RNAi is a phenomenon of the introduction of dsRNA into certain organisms and cell types causes degradation of the homologous mRNA.

RNAi was first discovered in the nematode Caenorhabditis elegans, and it has since been found to operate in a wide range of organisms. In recent years, RNAi has becomes an endogenous, efficient, and potent gene-specific silencing technique that uses double-stranded RNAs (dsRNA) to mark a particular transcript for degradation in vivo. RNAᵢ technology is disclosed, for example, in U.S. Pat. No. 5,919,619 and PCT Publication Nos. WO 99/14346 and WO 01/29058.

In one embodiment, the first and second modulators of the composition of the present invention are the same dsRNA. The dsRNA loaded microparticles would bind TLR and activate the TLR-dependent signaling pathway. Meanwhile, the dsRNA-loaded microparticles would be phagocytozed (by macrophages, DCs, monocytes) and activate FADD-dependent signaling pathway. Preferably, the dsRNA encodes an immune activator. Once inside the cell, the dsRNA is opened and translated to produce the immune activator that further activates the innate immune pathway. For example, the dsRNA may encode a component of the TLR pathway, such as TRIF or the IRAKs, which when introduced into cells would augment TLR-mediated activation of IFN-β and other innate immune responses.

In another embodiment, the first modulator is dsRNA and the second modulator is a component of the TLR pathway or a DNA molecule encoding a component of the TLR pathway.

In another embodiment, the first modulator is a component of FADD-dependent pathway, such as FADD, or a DNA molecule encoding a component of FADD-dependent pathway, and the second modulator is a dsRNA.

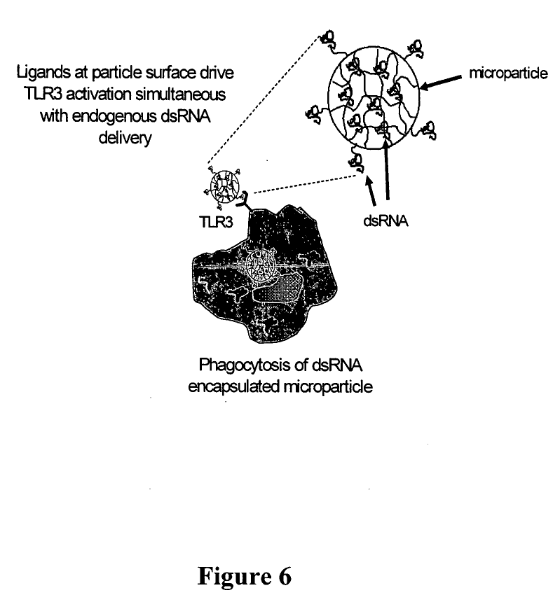

In another embodiment, the first and second modulators are dsRNAs or DNA molecules that encode any combination of antigenic products, components of the FADD pathway and/or products which will further enhance the immune response such as cytokines. The encoded products, once expressed inside the cell, would be processed via the endosomal pathway or the lysosomal pathways for MHC I or MHC II presentation on the cell surface, respectively. The dsRNA would activate the FADD-dependent, innate immune pathway. This scenario is schematically illustrated in FIG. 6. It is also likely that intracellular pathways will activate PKR, which has been proposed to play a role in facilitating the immune responses.

In yet another embodiment, the dsRNA containing microparticles can be further coated with a ligand for TLR3 to activate the TLR3 pathway or with heat shock proteins like gp96 or VSV G protein in order to target professional APCs such as DCs.

In another embodiment, the microparticles can be loaded with dsRNA representing silencing RNAi (siRNA) that can target genes for suppression following engulfment. In one embodiment, the siRNA suppresses the expression of a component of the FADD-dependent pathway, such as FADD, and down regulates antigen processing.

In another embodiment, the composition contains self-replicating RNA (replicon) based on positive stranded viruses (for example from pestivirus bovine diarrhea virus [BVDV] or alphaviruses). These RNA constructs are bicistronic consisting of 5′ terminal ORFs important for replicon IRES function and contains a natural start codon for translation. Foreign genes, such as those from influenza virus or other pathogens, can be placed downstream of a second IRES. The Replicon can be loaded onto chitosan particles and used to target antigen specific cells, ex vivo or in vivo. Once phagocytosed, replicons can reproduce themselves to high levels generating considerable dsRNA which will active the FADD/RIP-dependent pathway, functioning as an adjuvant, as described above. In addition, the replicon will translate the foreign gene to produce antigen that can be processed through the MHC class I or II pathways to stimulate CD4 and CD8 cells, specific for the antigen used. Replicons may be used to co-express pro-apoptotic molecules, such as caspases, or be co-loaded with purified pro-apoptotic molecules to induce cell death (or purified target antigens) which may enhance the antigen presenting process.

In another embodiment, the chitosan particles, loaded with intracellular or extracellular FADD or TOLL activating molecules such as dsRNA (as described above) can be co-loaded with purified antigens, such as from influenza virus or other pathogen related molecules, which may become processed to stimulate CD4, CD8 cells.

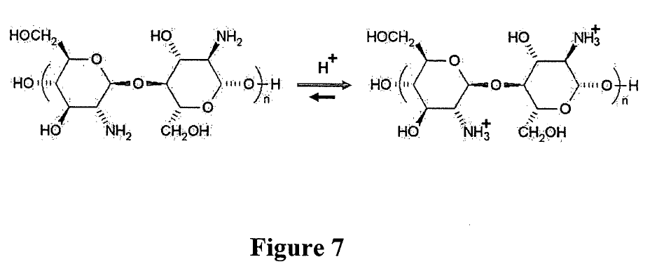

The present invention utilizes polycationic microparticles as the delivery system for the modulators of FADD-dependent and TLR-dependent pathway. Chitines and chitosanes (chitinosanes) are biodegradable polymers bearing multiple amino groups which acquire positive charges at neutral pH via association of hydrogen ion (FIG. 7). Comparing to microparticles made of other polymers, chitosan-based microparticles provide decreased agglomeration and better loading capacity for negatively charged molecules, especially nucleic acids. Protasan, a more purified version of chitosan, will be used interchangeably herein.

The microparticles of the composition of the present invention are designed to achieve a three-fold objective: delivery, temporary protection from the (primarily) enzymatic destruction in the body, and exposure or release of the loaded biomolecules (e.g., dsRNA, DNA, proteins and peptide, mode antigens etc.). Generally, the microparticle of the present invention are designed to release or expose the associated RNA/DNA/protein molecules quickly after entering the target cell to provide a vigorous immune response. In some applications, however, it may be desirable to release the associated molecules, such as cytokines, in a time-dependent manner.

Examples of cytokines include, but are not limited to, IL-12, IL-1α, IL-1β, IL-15, IL-18, IFNα, IFNβ, IFNγ, IL-4, IL-10, IL-6, IL-17, IL-16, TNFα, and MIF; as well as chemokines such as MIP-3α, MIP-1α, MIP-1β, RANTES, MIP-3β, SLC, fMLP, IL-8, SDF-1α, and BLC.

Chitosan microparticles can be produced using methods known in the art. Ravi Kumar et al. [Ravi Kumar, et al., Biomaterials, in press, 2003] demonstrated chitosan-stabilized PLGA cationic nanoparticles carrying DNA on their surfaces; the DNA was bound by simple mixing from watery solutions, thus, preserving integrity and conformation of the molecules. On the other hand, standard emulsion technique involving vigorous mixing with the carrier solution and emulgation schemes is also suitable for chitosan encapsulation of plasmids along with protein antigens [Thiele, et al., J. Controlled Release, 76:59-71, 2001]. These protocols can be utilized to prepare particles carrying various sets of cytokines or heat shock proteins together with dsRNA and/or DNA plasmids as discussed earlier.

Preferred methods for producing small microparticles (0.5-50 micron) are the micro gun and modified electrospray techniques, which are described in more details in the Examples. The “crumpled paper” shape enabled these particles with high surface areas for a high adsorption capacity for proteins and nucleic acids.

Chitosan polymers can be cross-linked with a crosslinking agent. Examples of crosslinking agents include, but are not limited to inorganic polyions, such as tripolyphosphate (TPP), sodium sulphate, and organic agents, such as glutaraldehyde and genipin.

Loading of nucleic acid and/or protein in chitosan particles can be achieved by direct admixing the nucleic acid and/or protein with chitosan during the fabrication of microparticles, externally saturating prefabricated microparticles with the nucleic acid and/or protein solutions, or a combination thereof. As shown in the examples, the external saturation method provides a higher loading efficiency than the direct admixing method. Combination of the two methods, however, showed an synergistic effect in enhancing the loading efficiency.

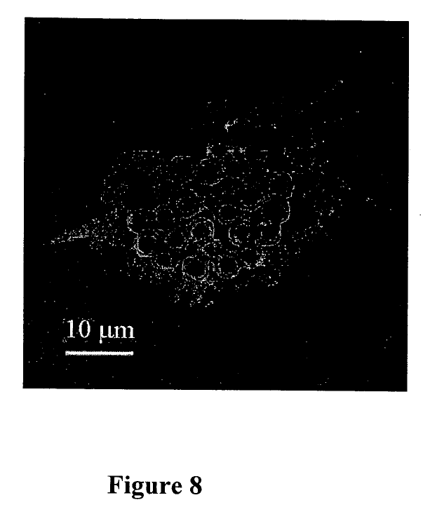

The microparticles of the present invention is small enough to be effectively phagocytosed and processed by APCs such as DCs and macrophages, as well as their precursors such as monocytes. In a preferred embodiment, the size of the microparticle is in a range from 0.5 to 70 microns, and more preferably from 0.5 to 20 microns. For example, FIG. 8 shows polystyrene beads, 4.5 μm, phagocytosed by monocyte-derived human DCs [(Thiele et al., J. cont. release 76:59-71 (2001)].

In another embodiment, the phagocytic properties of the microparticles is modified by using a mixture of hydrophilic chitosan polymer and one or more hydrophobic polymers. It is conceivable that modulation of the size and surface properties of the microparticles will become an extra leverage to control the relative efficacy of the activation of TRR/FADD pathways. By switching to the bigger and more hydrophilic particles unsuitable for phagocytosis, it is possible to expose the dsRNA signal molecules mostly to the TRR surfaces. Nano-sizes of chitosan particles may be produced using methods described in the examples. Larger chitosan particles, up to hundreds of micrometers, can be synthesized using the protocol of Denkbas et al. [Denkbas, et al., Reactive & Functional Polymers, 50:225-232, (2002)].

The release rates of nucleic acid and/or protein from chitosan particles can be controlled by adjusting several factors including the molecular weight of chitosan, the degree of deacetylation of chitosan, and the weight/charge ratios between chitosan and loaded biomolecules.

In one embodiment, the chitosan-based dsRNA/DNA/protein loaded micropaticles is encapsulated within a poly(lactide-co-glycolide) (PLGA) matrix/microparticles containing cytokines or antigens. PLGA has been shown to be biocompatible and it degrades to toxicologically acceptable lactic and glycolic acids that are eventually eliminated from the body. Release rates of the cytokines and the chitosan particles could be further controlled by adjusting the parameters involved for PLGA encapsulation, including monomer ratio/molecular weight of PLGA. Since chitosan/Protasan is hydrophilic, by encapsulating the chitosan/protasan particles in the more hydrophobic PLGA, the uptake of the particles into the cell across the cell membrane may be enhanced.

Alternatively, other types of polymers may be incorporated into the chitosan-based microparticles to achieve variable release profiles for the loaded biomolecules. For one example, a hydrophobic polymer, such as PLGA, can be blended with the more hydrophilic chitosan to form cationic PLGA particles. Other suitable polymers include, but are not limited to, poly(caprolactone), poly(oxybutirate).

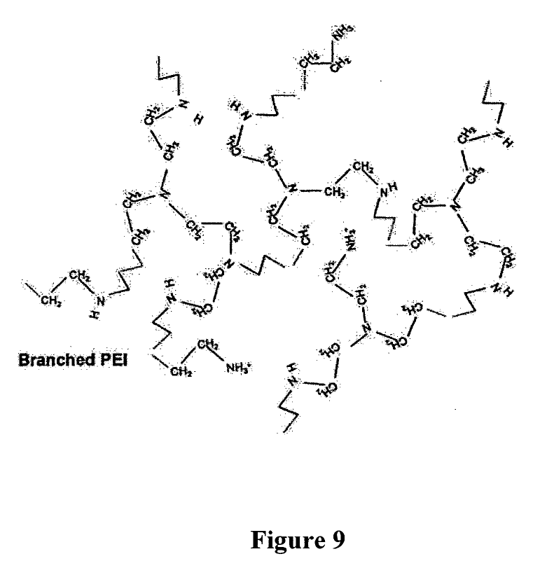

As another alternative, branched amphiphilic polyamine, poly(ethylene imine) (PEI) can be used instead of chitosan in combination with PLGA or other hydrophobic polymers (FIG. 9).

The addition of more hydrophobic domains to the chitosan particles could facilitate transport across the cell membrane. Another example includes forming porous particles by the addition of polyanionic sodium alginate to polycationic chitosan, as described by Liu et al. [Liu, et al.,k J. Controlled Release, 43:65-74, 1997]. By adjusting the ratio of the polymers, the pore size could be controlled and therefore the release rates of the dsRNA/cytokines from the particles.

The present invention also contemplates using cationic liposomes as a delivery vehicle. Cationic liposomes are good carriers for RNA, DNA and peptides [Honda, et al., J. Virol. Meth., 58:41-58, 1996; Nastruzzi, et al., J. Controlled Release, 68:237-249, 2000; Borgatti, et al., Biochemical Pharmacology, 64:609-616, 2002; Sioud, et al., Biochem. Biophys. Res. Commun., 312:1220-1225; 2003]. In general, liposomes offer a more adequate protection and better stabilization for RNA along with reasonable release kinetics. The considerations regarding phagocytosis, surface charge, and hydrophilicity remain applicable to liposomes. Using liposomes, dsRNA and its immunogenic substitutes such as poly(IC) or poly(ICLC) can be encapsulated in the vesicles and/or be attached to the surface. Phagocytosis of lipid cationic particles can be more pronounced than for hydrophilic colloid chitosan particles thanks to hydrophobic nature of the liposome surface. Special attention will be paid to controlling the appropriate 1-5 μm size of the lipid particle to enhanced phagocytosis.

In one embodiment, liposome carriers are used for stimulating the internal FADD pathway via phagocytosis, whereas large chitosan microparticles is used as surface carriers exposing dsRNA to the surface TLRs. Many combinations can be envisaged.

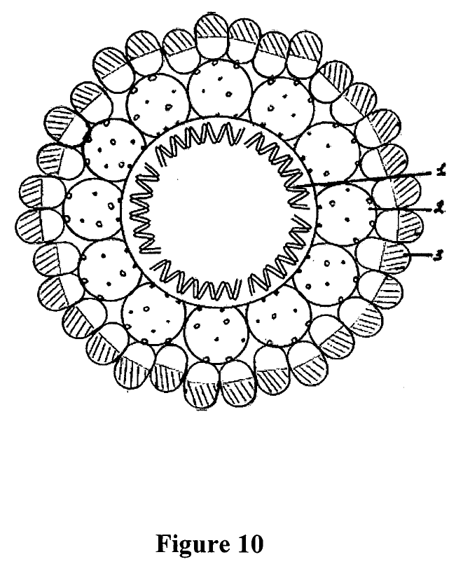

It is also possible to create a virus-like particle using a liposome-like structure carrying dsRNA in the center and protein HIV antigens on the surface [Karpenko, et al., Vaccine, 21: 386-302, 2003] (FIG. 10). FIG. 10 shows an artificial virus-like particles comprises (1) yeast dsRNA, (2) spermidine-polyglucin-glutathione conjugate, and (3) hybrid protein TBI-GST. In one embodiment, a reverse particle is created with the dsRNA on the surface and a protein antigen in the center.

In one embodiment, cross-signaling innate immune pathways is achieved with bacteria or fungus encapsulated in microparticles that undergo phagocytosis. Data indicates that the TLR pathway influences host defense against gram-positive bacteria while the imd (FADD) pathway exerts activity against gram-negative bacteria and fungus.

In another embodiment, cross-signaling innate immune pathways is achieved with a tumor antigen or a polynucleotide encoding a tumor antigen encapsulated in microparticles that under go phagocytosis.

The preferred embodiments of the compounds and methods of the present invention are intended to be illustrative and not limiting. Modifications and variations can be made by persons skilled in the art in light of the above teachings. It is also conceivable to one skilled in the art that the present invention can be used for other purposes of measuring the acetone level in a gas sample, e.g. for monitoring air quality. Therefore, it should be understood that changes may be made in the particular embodiments disclosed which are within the scope of what is described as defined by the appended claims.

Yet another aspect of the present invention relates to methods for preventing or treating various diseases using the immune activating composition of the present invention.

In one embodiment, the composition of the present invention is administered into a mammal for the prevention or treatment of infectious diseases. Examples of infectious diseases include, but are not limited to, diseases caused by viruses, such as Human immunodeficiency virus (HIV); influenza virus (INV); encephalomyocarditis virus (EMCV), stomatitis virus (VSV), parainfluenza virus; rhinovirus; hepatitis A virus; hepatitis B virus; hepatitis C virus; apthovirus; coxsackievirus; Rubella virus; rotavirus; Denque virus; yellow fever virus; Japanese encephalitis virus; infectious bronchitis virus; Porcine transmissible gastroenteric virus; respiratory syncytial virus; papillomavirus; Herpes simplex virus; varicellovirus; Cytomegalovirus; variolavirus; Vacciniavirus; suipoxvirus and coronavirus.

Further examples of infectious diseases include, but are not limited to, diseases caused by microbes such as Actinobacillus actinomycetemcomitans; Bacille Calmette-Gurin; Blastomyces dermatitidis; Bordetella pertussis; Campylobacter consisus; Campylobacter recta; Candida albicans; Capnocytophaga sp.; Chlamydia trachomatis; Eikenella corrodens; Entamoeba histolitica; Enterococcus sp.; Escherichia coli; Eubacterium sp.; Haemophilus influenzae; Lactobacillus acidophilus; Leishmania sp.; Listeria monocytogenes; Mycobacterium vaccae; Neisseria gonorrhoeae; Neisseria meningitidis; Nocardia sp.; Pasteurella multocida; Plasmodiumfalciparum; Porphyromonas gingivalis; Prevotella intermedia; Pseudomonas aeruginosa; Rothia dentocarius; Salmonella typhi; Salmonella typhimurium; Serratia marcescens; Shigella dysenteriae; Streptococcus mutants; Streptococcus pneumoniae; Streptococcus pyogenes; Treponema denticola; Trypanosoma cruzi; Vibrio cholera; and Yersinia enterocolitica.

In another embodiment, the composition of the present invention is administered into a mammal for the treatment of a cancer. Examples of cancer include, but are not limited to, breast cancer, colon-rectal cancer, lung cancer, prostate cancer, skin cancer, osteocarcinoma, and liver cancer.

The present invention further relates to a pharmaceutical composition comprising a FADD activator and a pharmaceutically acceptable carrier. The pharmaceutical composition may alternatively be administered subcutaneously, parenterally, intravenously, intradermally, intramuscularly, transdermally, intraperitoneally, or by inhalation or mist-spray delivery to lungs.

The carrier can be a solvent or dispersion medium containing, for example, water, ethanol, polyol (e.g., glycerol, propylene glycol, and liquid polyethylene glycol, and the like), or suitable mixtures thereof, and/or vegetable oils, solid microparticle or liposomes. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For parenteral administration in an aqueous solution, for example, the solution should be suitably buffered, if necessary, and the liquid diluent first rendered isotonic with sufficient saline or glucose. These particular aqueous solutions are especially suitable for intravenous, intramuscular, subcutaneous, intratumoral and intraperitoneal administration. In this connection, sterile aqueous media that can be employed will be known to those of skill in the art in light of the present disclosure. For example, one dosage may be dissolved in 1 ml of isotonic NaCl solution and either added to 1000 ml of hypodermoclysis fluid or injected at the proposed site of infusion, (for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. The microparticles of the present invention may also be administered into the epidermis using the Powderject System (Chiron, Corp. Emeryville, Calif.). The Powderject's delivery technique works by the acceleration of fine particles to supersonic speed within a helium gas jet and delivers pharmaceutical agents and vaccines to skin and mucosal injection sites, without the pain or the use of needles.

The compositions disclosed herein may be formulated in a neutral or salt form. Pharmaceutically-acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective. The formulations are easily administered in a variety of dosage forms such as injectable solutions, drug release capsules and the like.

The phrase “pharmaceutically-acceptable” or “pharmacologically-acceptable” refers to molecular entities and compositions that do not produce an allergic or similar untoward reaction when administered to a human. The preparation of an aqueous composition that contains a protein as an active ingredient is well understood in the art. Typically, such compositions are prepared as injectables, either as liquid solutions or suspensions; solid forms suitable for solution in, or suspension in, liquid prior to injection can also be prepared.

The term “therapeutically effective amount” as used herein, is that amount achieves, at least partially, a desired therapeutic or prophylactic effect in an organ or tissue. The amount of the FADD activator necessary to bring about prevention and/or therapeutic treatment of the FADD deficiency related diseases (such as infectious diseases and cancers) or conditions is not fixed per se. An effective amount is necessarily dependent upon the identity and form composition employed, the extent of the protection needed, or the severity of the diseases or conditions to be treated.

The present invention is further illustrated by the following examples which should not be construed as limiting. The contents of all references, patents and published patent applications cited throughout this application, as well as the Figures and Tables are incorporated herein by reference.

### EXAMPLE 1

### FADD Deficient Fibroblasts are Susceptible to Virus Infection

It is observed that murine embryonic fibroblasts (MEFs) that lacked FADD appeared super sensitive to virus infection [Balachandran, et al., J. Virol., 74:1513-1523, 2000]. To further examine this phenotype , a detailed analysis of virus replication in FADD+/− and FADD−/− MEFs using the IFN sensitive, prototypic rhabdovirus vesicular stomatitis virus (VSV) was performed.

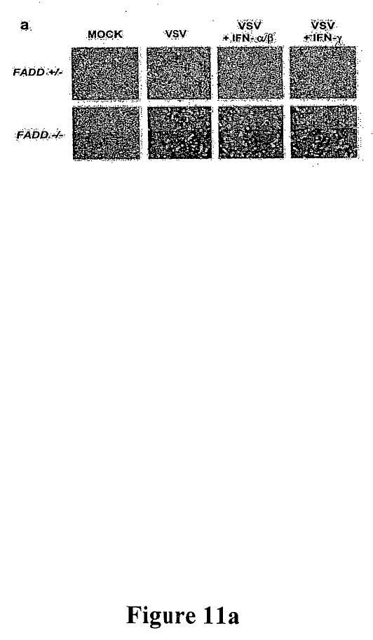

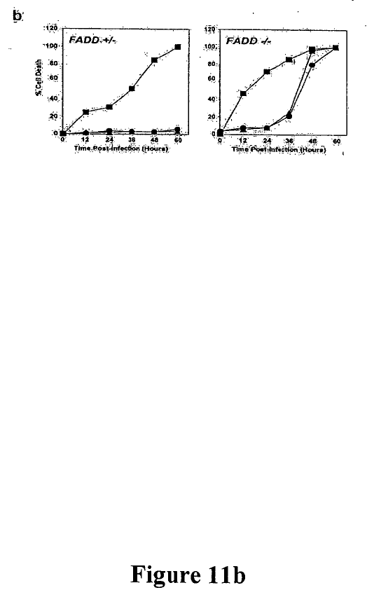

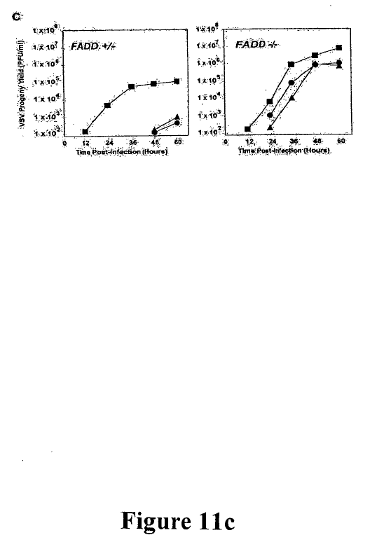

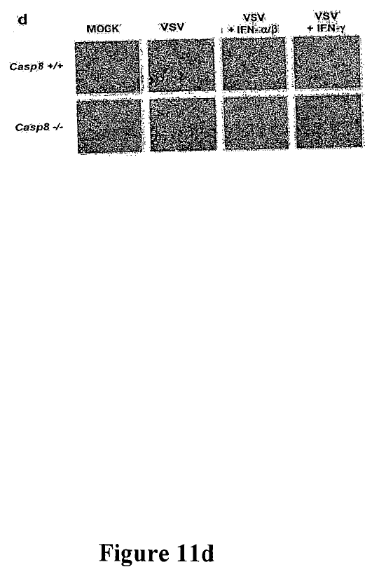

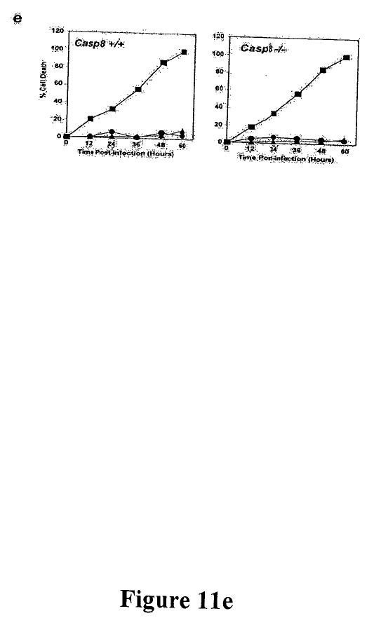

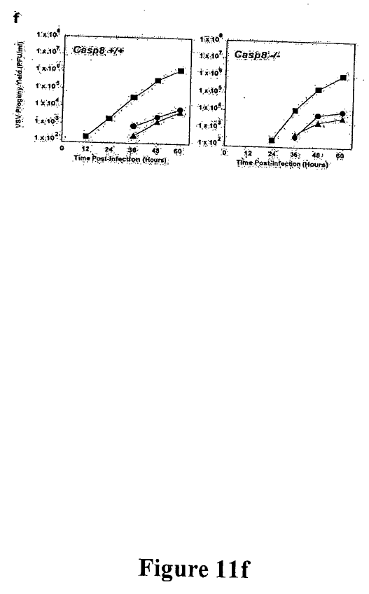

Briefly, FADD+/− and−/− MEFs were infected with VSV (MOI=5) in the presence or absence of 18 hours IFN α/β (500 U/ml) or IFN-γ (5 ng/ml) pretreatment, and photomicrographs were taken 48 hours post-infection. Following infection, observed that VSV replication was significantly augmented (>100-fold) in the FADD−/− MEFs, which concomitantly underwent rapid cytolysis, compared to their wild type counterparts (FIG. 11a).

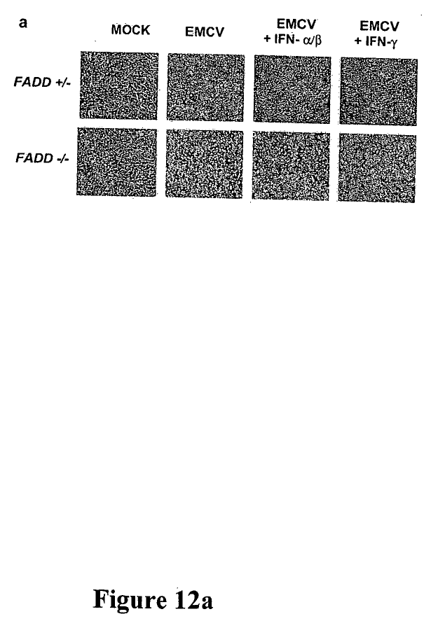

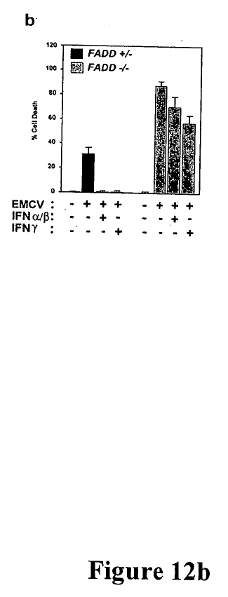

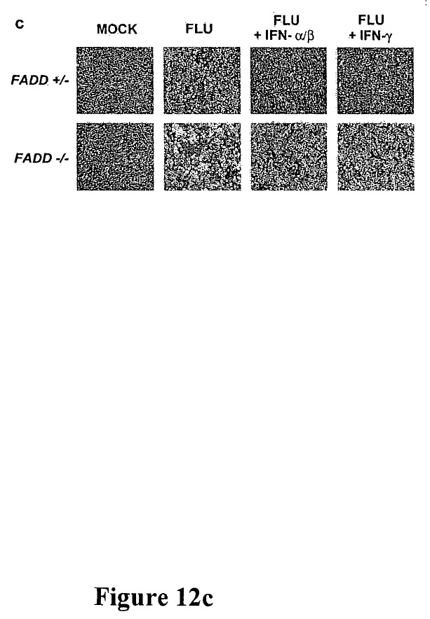

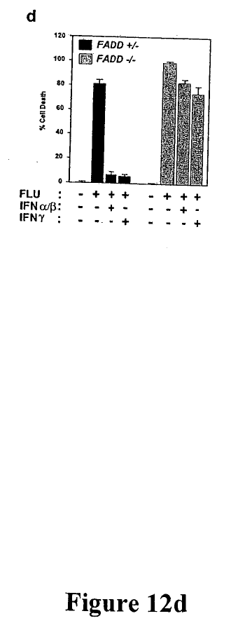

Moreover, Caspase-8+/+ and−/− MEFs were infected with VSV (m.o.i.=5) in the presence or absence of 18 hours IFN α/β (500 U/ml) or IFN-γ (5 mg/ml) pretreatment and photomicrographs were taken 48 hours post-infection. While treatment of MEFs with type I (α/β) or type II (γ) IFN for 12 hours was seen to exert significant antiviral activity in normal cells, as expected, these key antiviral cytokines only delayed the onset of viral replication in FADD−/− MEFs for up to 24 hours, whereupon virus replication proceeded unchecked (FIGS. 11b-e) (in FIG. 11c, at the indicated times post-infection, the medium was examined for progeny viral presence by standard plaque assay on BHK cells). The observed susceptibility to infection were not restricted to VSV, since cells lacking FADD were also sensitive to other virus types, including influenza virus (INV) and encephalomyocarditis virus (EMCV) (FIG. 12). Since these data indicate that FADD exerts a role in host defense against virus infection, a further investigation was conducted regarding whether the observed antiviral activity was governed through the canonical caspase 8-dependent signaling pathway [Muzio, et al., Cell, 85:817-827, 1996]. However, MEFs lacking caspase-8 exhibited no over susceptibility to VSV infection compared to control cells and retained the ability to respond to the antiviral effects of IFN (FIG. 11f). In FIG. 11f, IFN pretreatment efficiently inhibits VSV replication in both caspase-8+/+ and−/− EFs. Caspase-8 +/+ and−/− MEFs were infected with VSV (m.o.i.=5) in the presence or absence of 18 hours IFN α/β (500 U/ml) or IFN-γ (5 mg/ml) pretreatment. At the indicated times post-infection, the medium was examined for progeny virion presence by standard plaque assay on BHK cells. FIG. 11 demonstrates that FADD exerts antiviral activity through a caspase 8-independent pathway.

### EXAMPLE 2

### IFN Signaling is not Defective in the Absence of FADD

Since exposure to type I and II IFN was unable to fully protect FADD−/− MEFs from virus replication, it was plausible that effectual IFN signaling through the JAK/STAT pathway may require functional FADD for activity. To analyze the potential requirement for FADD in IFN-mediated signaling, FADD+/− and FADD−/− MEFS were treated with type I or II IFN and the expression and activity of the pivotal IFN signal transducer STAT1 was measured [Levy, et al., Nat. Rev. Mol. Cell. Biol., 3:651-662, (2002)].

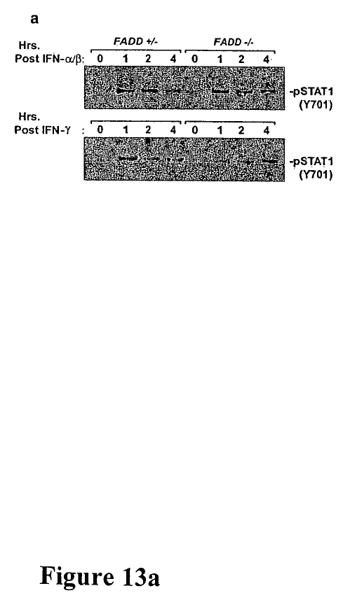

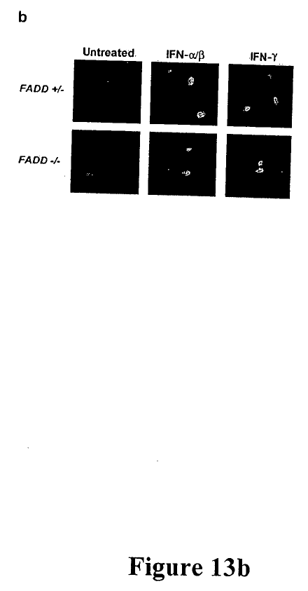

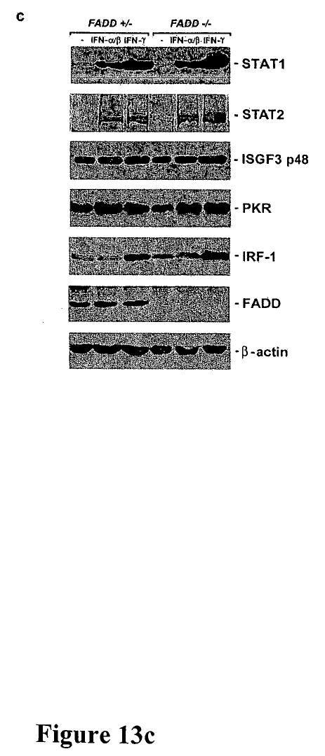

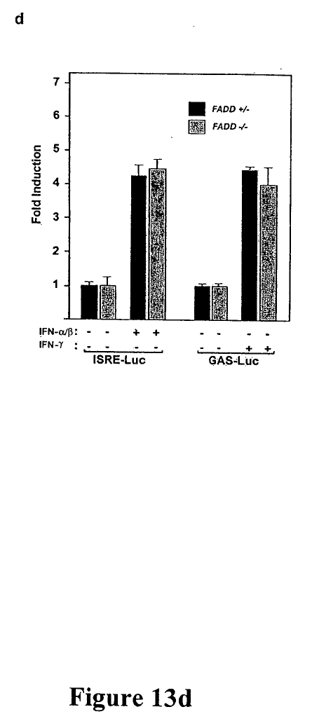

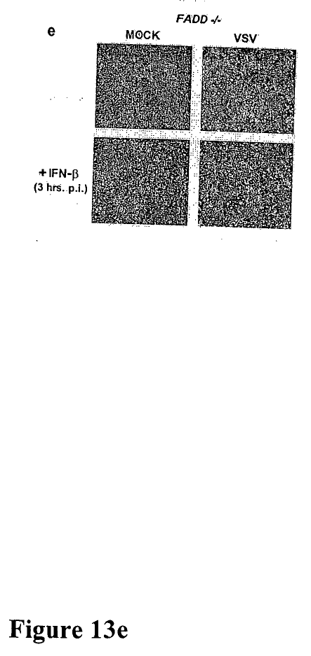

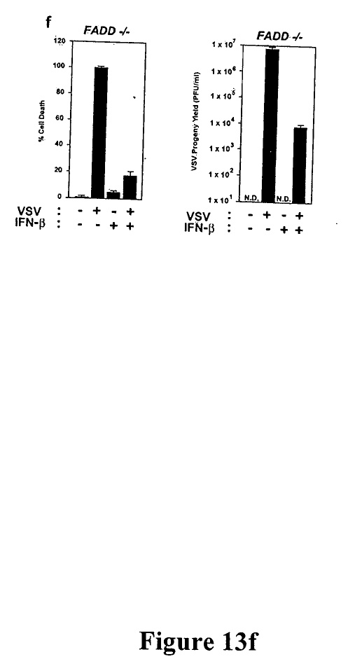

However, neither required phosphorylation of Y701 nor IFN-mediated signaling, nor the subsequent nuclear translocation of STAT1 appeared impaired in FADD−/− cells (FIGS. 13a-c). In FIG. 13a, FADD+/− and−/− MEFs were treated with either IFN α/β (500 U/ml) or IFN-γ (5 mg/ml) for the indicated times, and STAT1 phosphorylation status determined by immunoblotting using a STAT1 phospho-tryosine 701-specific antibody. In FIG. 13b, FADD+/− and−/− MEFs were transfected with a plasmid encoding a GFP-STAT1 fusion protein. 24 hours post-transfection, cells were treated with or without INF α/β (500 U/ml) or IFN-γ (5 mg/ml) for one hour and STAT1 localization was determined by GFP fluorescence microscopy. In FIG. 13c, FADD+/− and−/− MEFs were treated with or without IFN α/β (500 U/ml) or IFN-γ (5 mg/ml) for 18 hours. Lysates prepared from these cells were subject to immunoblot analysis for the indicated IFN-induced proteins.

Similarly, the expression of selected type I and II IFN-induced genes including IRF-1, PKR and STAT2 in response to IFN, appeared unaffected in FADD−/− cells [Der, et al., Proc. Natl. Acad. Sci. USA, 95:15623-15628, 1998]. Finally, luciferase reporter genes under control of type I IFN (ISRE) or type II (GAS) exhibited normal activity when transfected into FADD−/− cells treated with IFN (FIG. 10d). In FIG. 13d, FADD+/− and FADD−/− MEFs were transfected with plasmids expressing luciferase under the control of either the interferon stimulated response element (ISRE-Luc) or the interferon gamma activate sequence (GAS-Luc). 24 hours later, cells were stimulated with or without IFN α/β (500 U/ml) or IFN-γ (5 ng/ml) and luciferase activity measured 18 hours post treatment. These observations indicate that IFN signaling per se is not compromised in the absence of FADD.

### EXAMPLE 3

### Defective Induction of IFN-β by Intracellular dsRNA in the Absence of FADD

Despite the observations in Examples 1 and 2, it remained plausible that the anti-viral state initially established by 12 hours of exposure to exogenous IFN is short-lived and probably requires constant de novo synthesis following virus infection (FIG. 11a). For example, it was noted that constant supplementation of recombinant IFN-β to the medium of FADD−/− cells following VSV infection protected the cells from cytolysis (FIGS. 13e-13f). In FIG. 13e, IFN-treated FADD−/− MEFs were infected with VSV (m.o.i.=5) and subsequently treated with or without IFN-β (500 U.ml). Cells were photographed 48 hours post-infection. In FIGS. 13f, IFN-treated FADD−/− MEFs were infected with VSV (m.o.i.=5) and subsequently treated with or without IFN-β (500 U/ml). Cell viability and viral progeny yield were measured 48 hours post-infection.

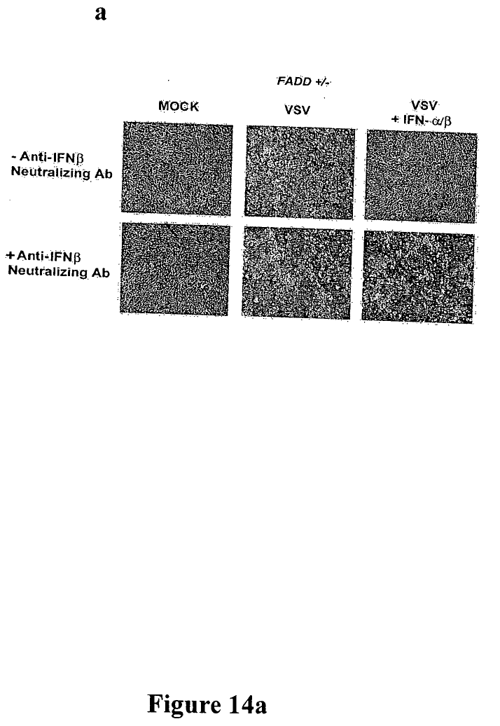

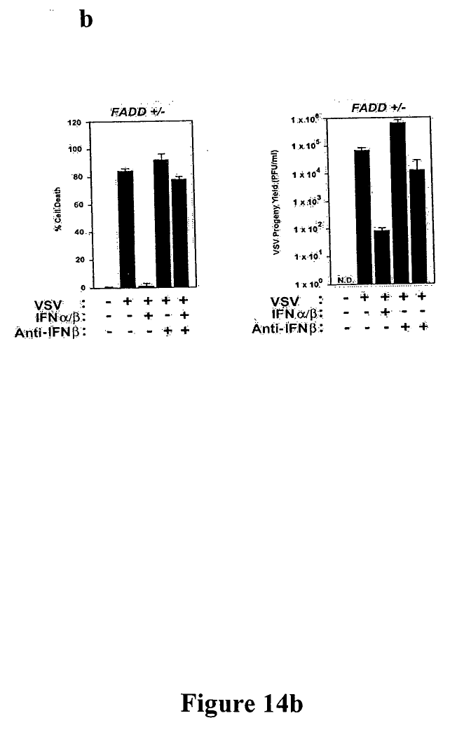

A constant requirement for IFN production was further emphasized by demonstrating that antibody-mediated neutralization of secreted IFN-β, following VSV infection of normal cells, re-invoked susceptibility to virus infection (FIGS. 14a and 14b). In FIG. 14a, FADD+/− cells were treated with IFN-α/β (500 U/ml), or were left untreated. These cells were subsequently infected with VSV (m.o.i.=5) and incubated for a further 48 hours in the presence or absence of neutralizing anti-IFN-β antiserum. Hotographs were taken 48 hours post infection (mag. 200×). In FIG. 14b, FADD+/− cells treated as in FIG. 14a were examined for VSV progeny yield or cell viability by Trypan Blue exclusion.

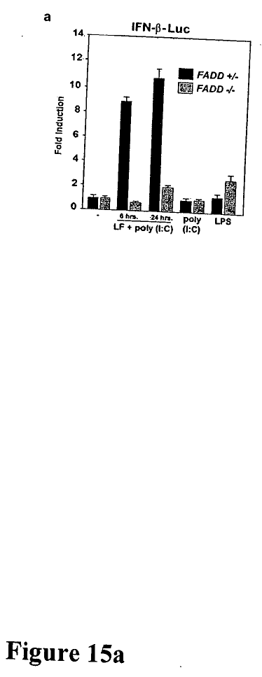

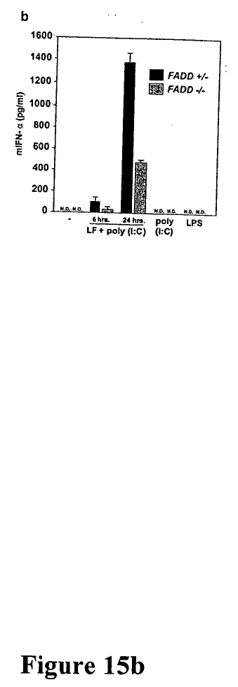

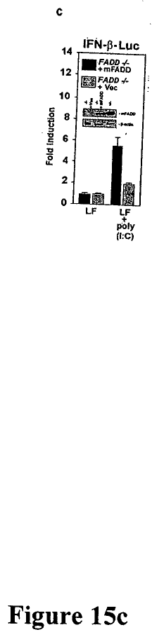

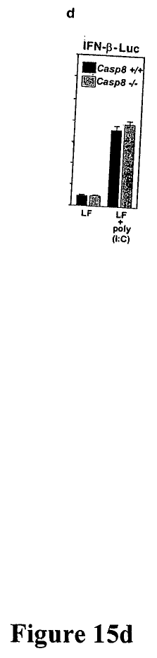

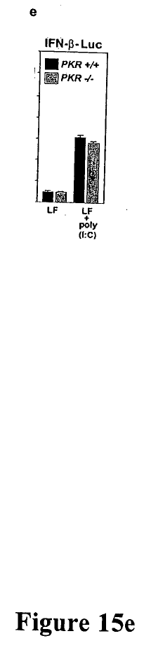

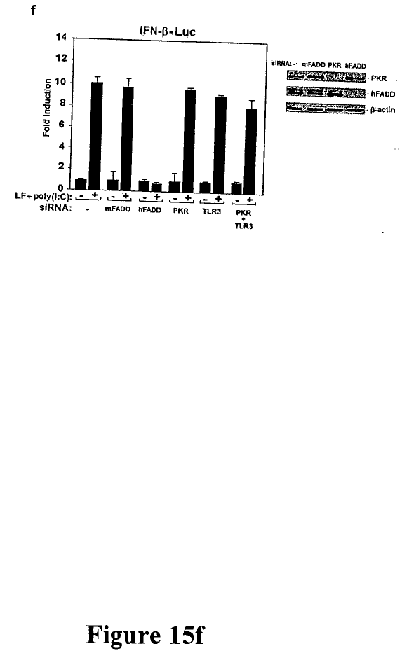

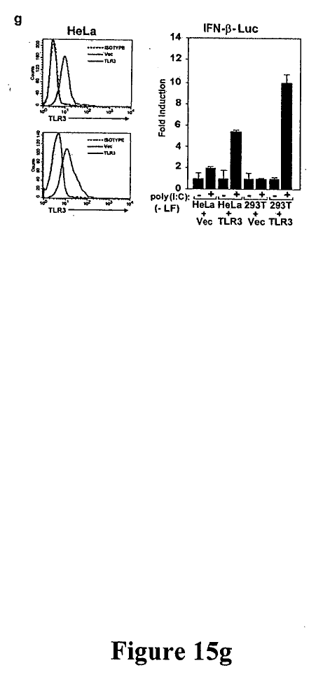

These analyses indicated that a defect in the production of IFN-β following virus infection might explain the susceptibility of FADD−/− cells to virus infection. To examine this possibility, FADD+/− and FADD−/− cells were transfected with a luciferase reporter construct under control of an IFN-β promoter and subsequently administered poly(IC), a synthetic mimetic of viral dsRNA, thought to be the primary trigger of IFN production following virus infection [Kerr, et al., Philos. Trans. R. Soc. Lond. B Biol. Sci., 299:59-67, 1982]. Briefly, FADD+/− and FADD−/− MEFs were transfected with a plasmid encoding luciferase under control of the human IFN-β promoter (IFN-β-Luc). 24 hours later, these cells were treated with poly(IC) alone [50 μg/ml], transfected poly(IC) [4 mg/ml in Lipofectamine2000) or LPS (5 ml/ml) and luciferase activity measured 6 or 24 hours post treatment. Data indicated that transfected poly(IC) triggered robust (>10 fold) induction of the IFN-β promoter in FADD+/− cells but not in cells lacking FADD (FIG. 15a).

Further, FADD+/− and FADD−/− MEFs were treated with poly(IC) alone [50 μg/ml], transfected poly(IC) [4 mg/ml mg/ml in Lipofectamine2000) or LPS (5 mg/ml) and IFN-α in supernantants measured by ELISA (PBL) 6 or 24 hours post treatment. This defect in IFN production in response to transfected dsRNA and VSV was confirmed in FADD deficient MEFs following ELISA specific for IFN production (FIG. 15b and data not shown).

In FIG. 15c, FADD−/− MEFs were transfected with either empty vector (pcDNA3Neo) or pcDNA3Neo encoding full length mFAD, along with IFN-β-Luc. 24 hours later, cells were transfected with poly(IC) [4 mg/ml in Lipofectamine 2000] and luciferase activity measured 6 or 24 hours later. Result shows that the restoration of poly(IC)-induced activation of IFN-β could be achieved by transiently transfecting murine (m) FADD back into FADD−/− MEFs (FIG. 15c).

Furthermore, Caspase-8+/+ and PKR−/− cells were transfected with IFN-β-Luc. 24 hours later, these cells were transfected with poly(IC) [4 mg/ml in Lipofectamine 2000] and luciferase activity measured after 6 hours. The defect in poly(IC) induced IFN-β induction was not apparent in caspase-8 deficient MEFs (FIG. 15d). Since the induction of IFN-β was not strongly observed using non-transfected, exogenous poly(IC) alone, it can be concluded that the observed IFN-induction in normal MEFs almost certainly involves intracellular dsRNA-recognition components and was TLR 3 independent (FIGS. 15a-b). However, the signaling did not appear to involve the dsRNA-activated molecule PKR, since MEFs lacking this kinase retained IFN-β induction in response to transfected dsRNA (FIGS. 15e-f). In FIG. 15e, PKR+/+ and PKR−/− MEFs were transfected with IFN-β-Luc. 24 hours later, these cells were transfected with poly(IC) [4 mg/ml in Lipofectamine 2000] and luciferase activity measured after 6 hours.

In FIG. 15f, RNAi-mediated knockdown of FADD, but not PKR or TLR3 abolishes intracellular dsRNA signaling. HeLa cells were treated with siRNA sequences from mFADD, hFADD, PKR, or TLR3, and knockdown of the respective gene products confirmed by immunoblotting and RT-PCR (data not shown). These cells were then transfected with IFN-β-Luc, and subsequently transfected with poly(IC) (4 mg/ml in Lipofectamine 2000). Luciferase activity was measured 6 hours later.

Further, PKR-deficient mice infected with VSV, retained the robust ability to induce IFN-β (FIG. 15). Neither could the observed virus/dsRNA-mediated activity be explained through TLR3 signaling. For example, we found little TLR3 activity in MEFs, HeLa and 293T cells (FIGS. 15f-g). iRNA -mediated depletion of only FADD, and not PKR or TLR3 (or both simultaneously), in HeLa cells resulted in an almost complete abrogation of IFN-β promoter activity, in response to transfected poly(IC) (FIG. 15g). In FIG. 15g, HeLa or TLR3 were transfected with a plasmid encoding TLR3, and expression was confirmed by flow cytometry (left). These cells were subsequently transfecetd with the IFN-β-Luciferase construct, and subsequently either treated with poly(IC) alone [50 μg/ml], or were transfected with poly(IC) [4 mg/ml in Lipofectamine 2000], and luciferase activity measured 6 hours later.

These data would thus infer a TLR 3/PKR independent dsRNA signaling pathway in eukaryotic cells. To further dissect the nature of FADD-mediated antiviral activity, the ability of VSV or poly(IC) to individually activate each of apical signaling cascades involved in IFN-β promoter activation, i.e. NF-κB, AP-1 and IRF-3 was examined [Agalioti, et al., Cell, 103:667-678, 2000; Thanos, et al., Cell, 83:1091-1100, 1995]. Using reporter constructs responsive to each of these three transcription factors, very little IRF3 activity and modest AP-1/NF-κB activity were detected in normal MEFs in response to transfected dsRNA. The result is probably due to the inherent difficulty in transfecting these cell types and the weak activity of the individual promoters (data not shown). However, robust signaling of NF-κB and AP-1 in HeLa cells was observed in response to transfected poly(IC), which appeared clearly compromised in the absence of FADD (FIG. 13f). Thus, FADD-mediated signaling involves activation of NF-κB and AP-1.

### EXAMPLE 4

### Normal Toll Receptor Signaling in the Absence of FADD

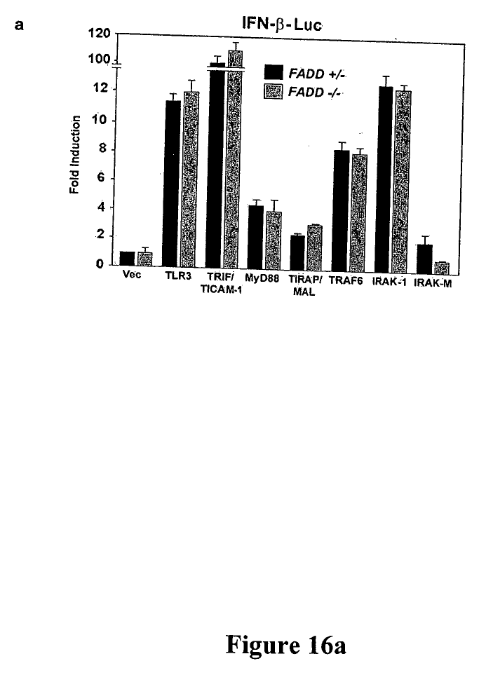

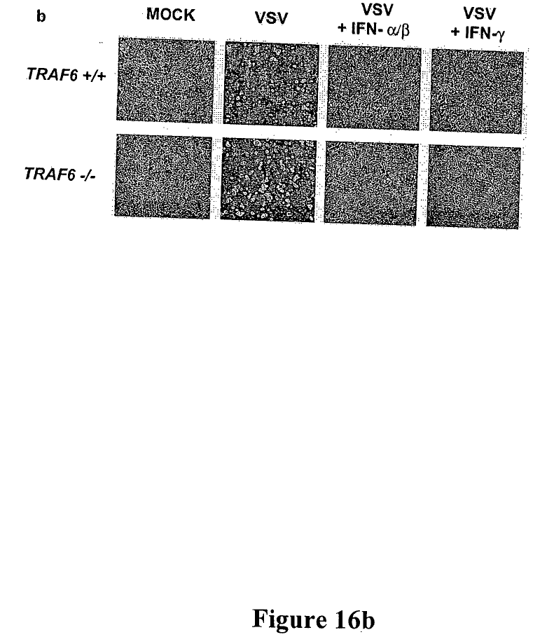

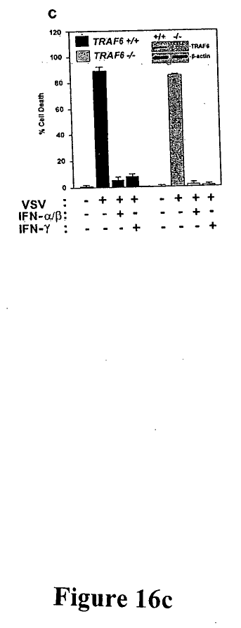

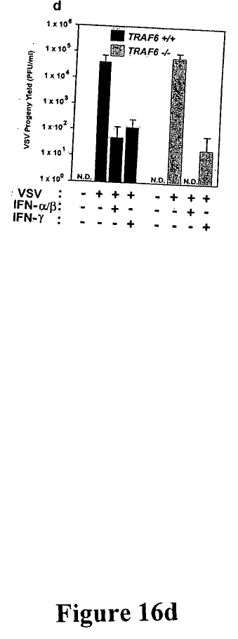

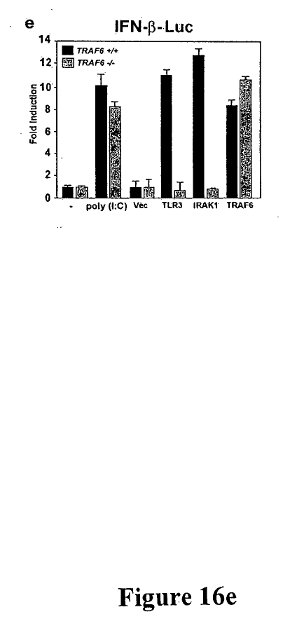

It has recently been shown that TLR3 is involved in the recognition of extracellular dsRNA, which can lead to the induction of IFN-β through activation of the IRAK family members and TRAF6 [Alexopoulou, et al., Nature, 413:732-738, 2001]. To further clarify whether FADD plays a role in TLR-mediated signaling, FADD+/− or FADD−/− MEFs were transfected with an IFN-β-luciferase reporter construct and plasmids encoding various components of the TLR signaling pathway (such as TLR3, IRAK-M, IRAK-1, MyD88, TIRAP/MAL, TRIF/TICAM-1, and TRAF6), many of which have been shown to induce IFN-β gene expression following transient overexpression [Akira, J. Biol. Chem., 278:38105-38108, 2003]. However, no abrogation in TLR-mediated induction of IFN-β was observed in FADD deficient cells (FIG. 16a). In FIG. 16a, plasmids encoding the indicated TLR signaling components were co-transfected with IFN-β-Luc intor FADD+/− and FADD−/− MEFs and luciferase activity measured 24 hour post-transfection. Moreover, TLR3, TRIF and IRAK1 overexpression was able to stimulate a >10-fold increase in IFN-β promoter activity in both FADD containing and lacking MEFs (data not shown). These results were verified by demonstrating that TRIF deficient MEFs retained the ability to induce IFN-β in response to transfected dsRNA, unlike FADD−/−.

To further confirm these findings, the role of TRAF6 in anti-viral immunity was examined, a key downstream intermediary of TLR activity that is responsible for modulating NF-κB/AP-1 activation of IFN-β [Wu et al., Bioessays, 25:1096-1105 (2003)]. Accordingly, TRAF6+/+ and TRAF6−/− fibroblasts were infected with VSV (MOI=5) in the presence or absence of 18 hours IFN α/β (500 U/ml) or IFN γ (5 ng/ml) pretreatment. However, unlike FADD−/− cells, it was found that exposure to IFN efficiently protected TRAF6−/− MEFs against VSV infection similar to wild type control cells (FIG. 16b) (Photomicrographs were taken 48 hours post-infection). Next, the ability of intracellular poly(IC) to activate the IFN-β promoter in TRAF6−/− MEFs was examined. TRAF6+/+ and TRAF6−/− EFs were infected with VSV (MOI=5) in the presence or absence of 18 hours IFN α/β (500 U/ml) or IFN γ (5 ng/ml) pretreatment. Cell viability was determined by Trypan Blue exclusion analysis 48 hours post infection. This analysis indicated that transfected poly(IC) retained the ability to activate IFN-β in the absence of TRAF6, indicating that this adaptor molecule probably does not play a role in FADD-mediated dsRNA-intracellular signaling (FIGS. 16c-d).

Furthermore, it was not observed a significant role for FADD in other TLR pathways (data not shown). Demonstrating that TLR3 and IRAK1 were unable to mediate IFN-β induction in the absence of TRAF6−/− would collectively indicate that FADD functions independent of the TLR/TRAF6 and TRIF pathways (FIG. 16e). FIG. 16e shows that IFN Pretreatmerit protects TRAF6−/− MEFs from VSV. TRAF6+/+ and TRAF6−/− EFs were infected with VSV (m.o.i.=5) in the presence or absence of 18 hours IFN α/β (500 U/ml) or IFN-γ (5 ng/ml) pretreatment. In this experiment, the medium was examined for progeny virion presence 48 hours post-infection by standard plaque assay on BHK cells. Normal intracellular dsRNA signaling in the absence of TRAF6. TRAF6+/+ and TRAF6−/− EFs were transfected with IFN-β-Luc for 24 hours, and subsequently transfected with poly(IC) (4 mg/ml in Lipofectamine 2000) for 6 hours, after which luciferase activity was measured. TLR3 and IRAK-1 require TRAF6 for IFN-β gene induction. TRAF6+/+ and TRAF6−/− EFs were transfected with plasmids encoding TLR3, IRAK-1 or TRAF6, along with IFN-β-Luc, and luciferase activity was measured 24 hours post-transfection.

### EXAMPLE 5

### A Mammalian IMD-Like Pathway Confers Anti-Viral Innate Immunity

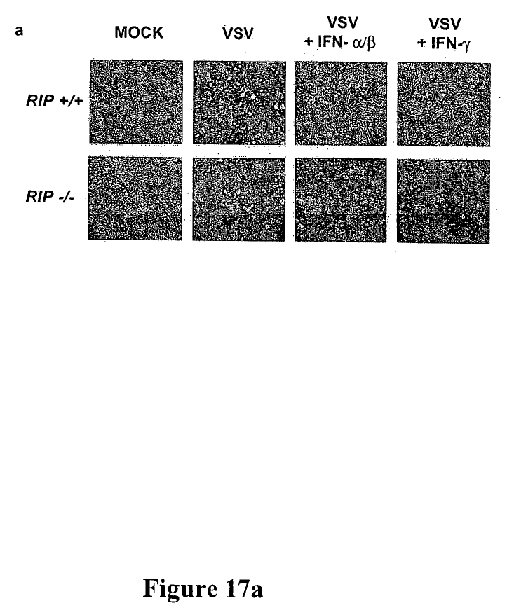

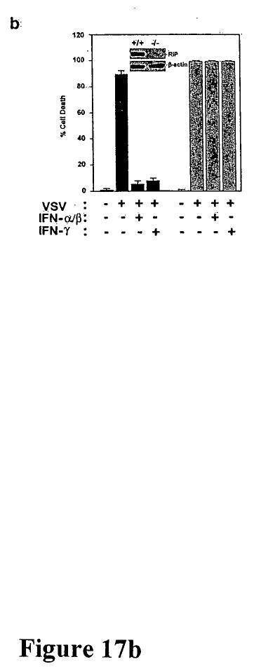

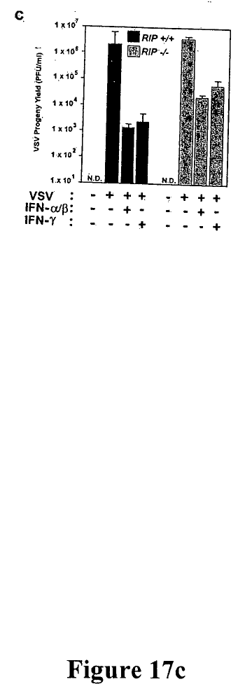

Data indicate that FADD plays a key role in innate immunity to virus infection and is independent of the TRAF6 mediated TLR3 pathway. Further, FADD has recently reported to be involved in the innate immune response to bacterial infection in Drosophila [Leulier, et al., Curr. Biol., 12:996-1000, 2002; Naitza, et al., Immunity, 17:575-581, 2002]. In these organisms, the immunodeficient (imd) gene product, a Drosophila homologue of the mammalian death domain containing kinase, RIP, associates with dFADD to trigger activation of an NF-κb related pathway and subsequent induction of antibacterial genes [Hoffmann, Nature, 426:33-38, 2003]. To determine if an IMD-like pathway, involving FADD, exists in mammalian cells, IFN-treated or untreated RIP−/− MEFs were infected with VSV (MOI=5). FIG. 17a shows VSV-induced cytolysis in RIP−/− cells but not controls. In this experiment, the VSV-induced cytolysis was observed even in the presence of IFN, similar to the FADD−/− MEFs.

As shown in FIGS. 17b and 17c, approximately, ten- to fifty-fold more VSV was generated in IFN-treated RIP−/− MEFs compared to wild type MEFs, with similar results being obtained following infection with influenza virus or EMCV. In FIG. 17b, FADD+/− and FADD−/− EFs were infected with VSV (m.o.i.=5) in the presence or absence of 18 hours IFN α/β (500 U/ml) or IFN-γ (5 ng/ml) pretreatment. At the indicated times post-infection, the medium was examined for progeny virion production. In FIG. 17c, RIP+/+ and −/− EFs were infected with VSV (m.o.i.=5) in the presence or absence of 18 hours IFN α/β (500 U/ml) or IFN-γ (5 ng/ml) pretreatment. At the indicated times post-infection, the medium was examined for progeny virion production.

In addition, RIP-deficient MEFs, as well as HeLa cells in which RIP expression was abrogated using RNAi, exhibited a selective and profound inability to respond to intracellular dsRNA-mediated signaling of the IFN-β promoter (FIGS. 17d-e). In FIG. 14e, RIP+/+ and−/− EFs (left) or HeLa cells in which RIP was specifically knocked down by RNAi (right) were transfected with IFN-β-Luc for 24 hours, and subsequently transfected with poly(IC) (4 mg/ml in Lipofectamine 2000) for 6 hours, after which luciferase activity was measured. In FIG. 17f, RIP+/+ and−/− EFs were transfected with plasmids encoding TLR3, IRAK-1 or TRAF6, along with IFN-β-Luc, and luciferase activity was measured 24 hours post-transfection. These results show that TLR3, IRAK1, TRAF6 and TRIF were able to robustly induce IFN-β promoter activity, following transient overexpression in RIP−/− MEFs, providing further evidence that intracellular and extracellular dsRNAs utilize divergent signaling pathways to induce IFN-β.

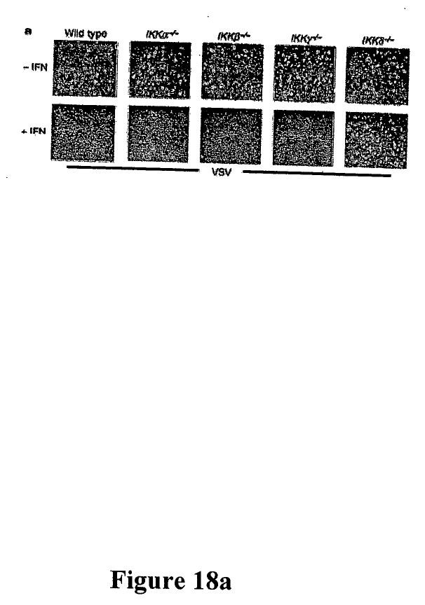

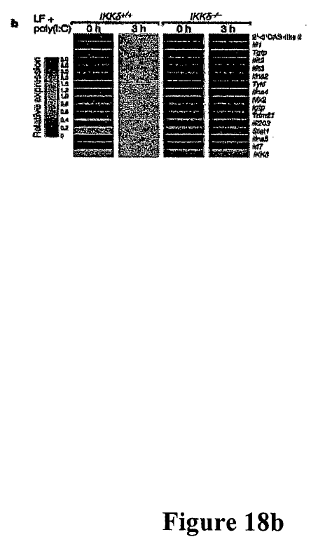

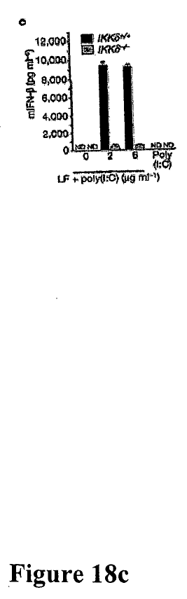

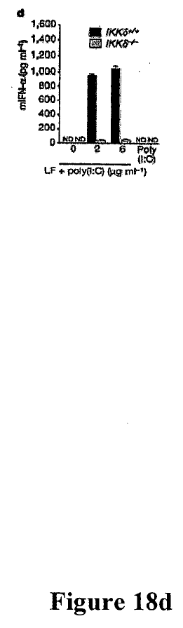

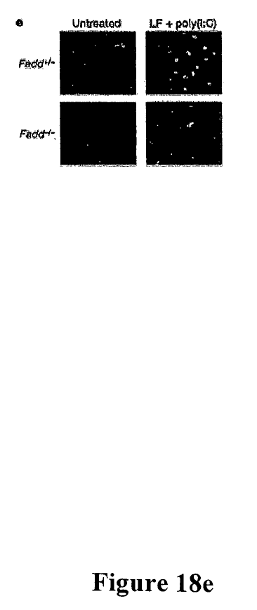

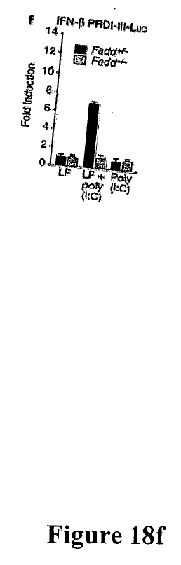

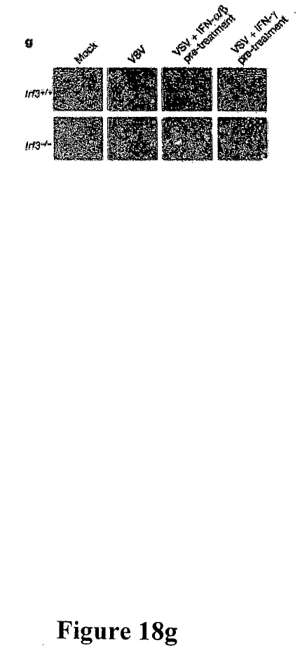

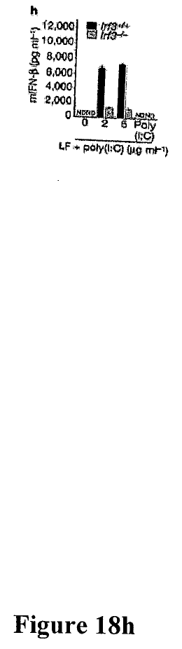

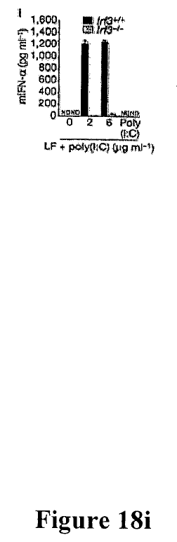

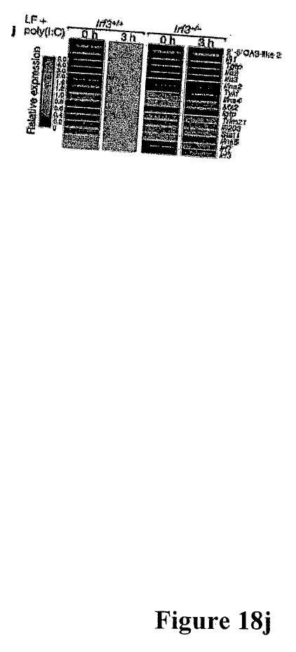

In Drosophila, imd and dFADD are required to stimulate the induction of antimicrobial gene expression through activation of the NF-κB homologue Relish via an I-κB kinase (IKK) complex comprised of IKK-β /IRD5 and IKK-γ /Kenny. In mammalian cells, induction of IFN-β also involves activation of NF-κB, as well as IRF-3. In FIG. 18a, wild-type or IKK-α-, IKK-β-, IKK-γ- and IKK-δ-deficient MEFs were infected with VSV (MOI ¼ 10) with or without IFN-α/β (100 Uml21) pre-treatment. Result shows that pre-treatment with IFN was able to effectively protect MEFs lacking IKK-α, -β or -γ against virus infection (FIG. 18a). This study was complemented by examining MEFs lacking Tank-binding kinase 1 (TBK-1)/IKK-δ, as this molecule seems to be the primary IRF-3 kinase in MEFs. This experiment revealed that, similar to FADD−/− and RIPk1−/− fibroblasts, TBK-1/IKK-δ-deficient cells are not protected against virus replication and cytolysis even after pre-treatment with IFN (FIG. 18a).

Similar to FADD−/− cells, these results could be explained by a defect in type I IFN induction in TBK-1/IKK-δ-deficient MEFs. DNA microarray, RT-PCR and ELISA analyses confirmed a severe impairment of dsRNA-responsive induction of type I IFN, as well other antiviral genes, in the absence of TBK-1/IKK-δ (FIGS. 18b-d). These results indicate that FADD may mediate its effects predominantly through TBK-1 activation of IRF-3. Accordingly, IRF-3 translocation, which occurs after phosphorylation by TBK-1/IKK-δ and IKK-1, was found to be defective in FADD−/− cells after treatment with transfected dsRNA (FIGS. 15e and f). Notably, Irf3−/− MEFs were not fully protected against virus infection after exposure to type I or II IFNs (FIG. 18g). Similarly, DNA microarray, RT-PCR, ELISA and RNA interference analyses confirmed a defect in the ability of intracellular dsRNA to induce type I IFN production in IRF3−/− MEFs (FIGS. 18h-j).

These results suggest that viral dsRNAs are recognized by an intracellular receptor molecule, which may recruit FADD and RIPI into an ‘innateosome’ complex to regulate TBK-1/IKK-δ-mediated activation of IRF-3. It was shown that the loss of FADD or RIP 1 leads to a defect in IFN-β production and a consequent lag in the production of IRF-7 and members of the IFN-α family, which are necessary for fortification of the antiviral state 3. It is also noteworthy that TBK-1/IKK-δ-deficient MEFs display a more profound defect in the induction of type I IFNs in response to dsRNA stimulation than either FADD-deficient or RIP1-deficient MEFs alone, plausibly suggesting that intracellular dsRNA-activated complexes retain some activity in the absence of FADD, or that alternative FADD-independent intracellular signaling cascades converge on TBK-1/IKK-δ. This RIP1/FADD/TBK-1 (RIFT) pathway seems to be largely independent of TLR3, PKR, TRIF/TICAM-1 or TRAF6, and is in agreement with other findings suggesting the existence of alternative intracellular, dsRNA-activated signal transducers, such as the DExD/H helicase RIG-I.

### EXAMPLE 6

### The Role of FADD in Mammalian Responses to Bacterial Infection

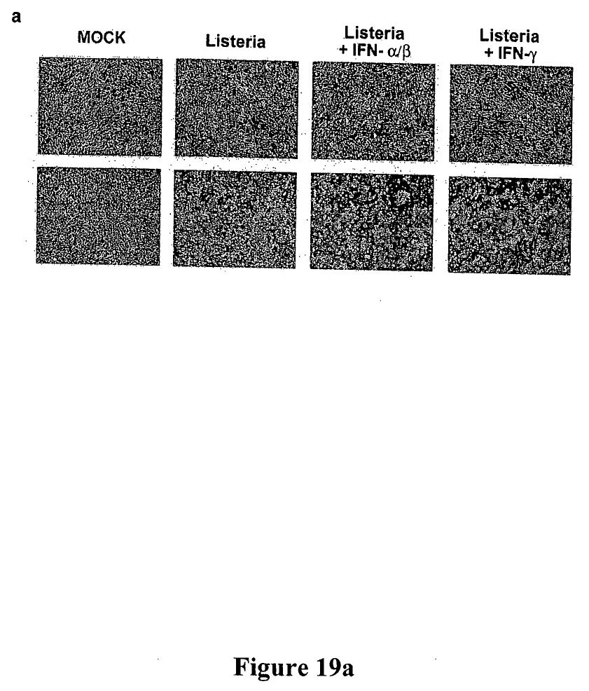

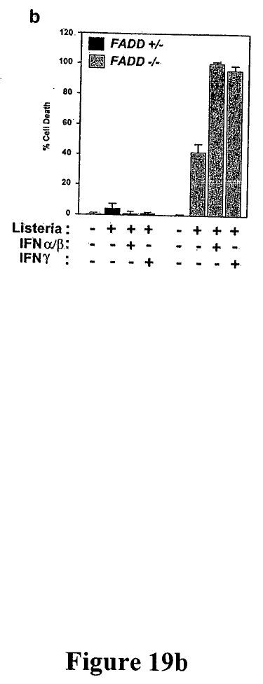

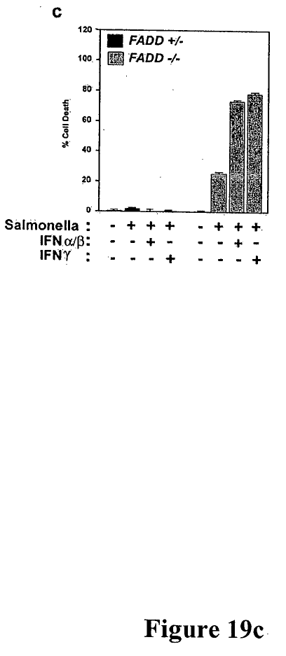

The role of the imd pathway in Drosophila is reported to involve the response to gram-positive bacteria infection and the existence of an antiviral pathway has not yet been determined. Whether innate responses to intracellular bacterial infection that was effected by loss of FADD or RIP in mammalian cells was examined and as shown in FIG. 19. Briefly, FADD+/−, FADD−/− or RIP −/− MEFs were treated with or without IFN-α/β or IFN γ for 18 hours and infected with 5 μl of an over night culture of the intracellular gram-positive bacteria, Lysteria monocytogenes and incubated for a further 24 hours in medium containing 10 ig/ml gentamycin (FIGS. 19a and 19b); or infected with 50 μl of an over night culture of the gram-negative Salmonella typhimurium, and incubated for a further 48 hours in medium containing 10 ig/ml gentamycin (FIG. 19c). Significantly, it was observed dramatic cell death occurring in the FADD and RIP deficient fibroblasts following exposure to bacterial infection. This effect was accompanied by an increase in bacteria replication. This data indicates that similar to insect cells, the FADD pathway is important in innate immunity to bacteria infection.

### EXAMPLE 7

### Prefabrication of Chitosan Particles with Large Surface Area

Either a Micro Spray Air Gun or Electrospray methods were used for chitosan microparticles prefabrication. In the Micro Spray Air Gun method the chitosan solution was dispersed turbulently to the smallest dimensions possible for the gun. The sizes of the particles were controlled mostly by the surface tension and were in the range from ˜20 to 100 microns.

Electrospray is a method of electrostatic atomization of liquids. An electrostatic field compels a fluid to jet out of a capillary electrode towards the receiving counter electrode. Secondary stepwise splitting and pulverization of droplets due to Coulomb repulsion produces plume of fine microdroplets. To prevent surface film formation, a Modified Electrospray method was set up.

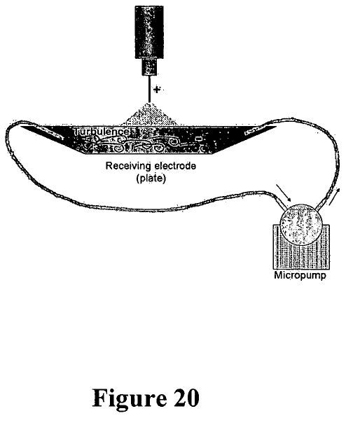

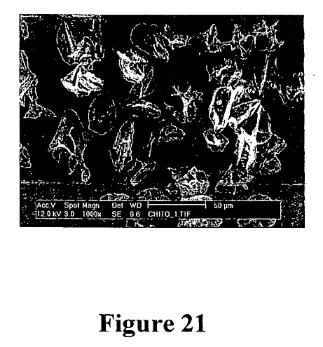

Electrospraying of chitosan onto a still surface of the crosslinking solution (tripolyphosphate, TPP) resulted in the formation of thin surface film of the stabilized chitosan instead of microparticles, due to extremely fine and homogeneous pulverization of the chitosan solution. To prevent this undesirable effect, a turbulent recirculation of the crosslinking solution was devised (FIG. 20). A circulation micropump provided open loop circulation of the TPP solution in the receiving electrode plate essential for disruption of the film. The modified Electrospray unit was used to pulverize 1%, 1.5% and 2% chitosan solutions in water and 25% ethanol. A 25G stainless steel capillaries (EFD) worked as pulverizing electrodes, while a 10 inch stainless steel plate containing 100 ml of 10% TPP solution was used as the receiving counter electrode. Electrospray with the turbulent agitation of the crosslinking TPP solution created microparticles smaller than that obtained using the Micro Spray Gun plume mode: the sizes have occurred distributed from ˜5 to ˜50 microns (FIG. 21).

Chitosan droplets prefabricated by the modified electrospray method were of an around micron size: significant 90 degree scattering of red laser beam by the Electrospray plume was observed indicating to the droplet sizes comparable with the wavelength of light. The larger apparent size observed for the dry particles is explained by their subsequent transformation: upon contact with the TPP solution the surface tension forces spread the microdroplets into the ultrathin sheets on the surface of TPP. This unusual shape was well seen in the microscope. Upon freeze drying the microsheets shrank into shapes resembling crumpled paper, and never spread again after re-suspending. The above described methods of prefabrication microparticles produce wide range of the microparticles with large surface areas. The particles of the smaller size could be engulfed by dendritic cells. On the other hand, the large surface area of these particles provides a significant advantage for external saturation with nucleic acids and proteins.

### EXAMPLE 8

### Chitosan Particles Loaded with polvinosinic-polycytidylic Acid

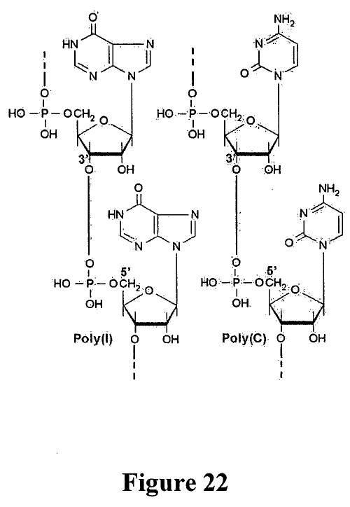

Polyinosinic-polycytidylic acid, poly(IC) is an interferon (IFN) inducer consisting of a synthetic, mismatched double-stranded RNA. The polymer is made of one strand each of polyinosinic acid and polycytidylic acid (FIG. 22).

Being a polyanion, poly(IC) is strongly adsorbed by the polycationic chitosan. Two methods of manufacturing poly(IC)-loaded chitosan particles were used: Admixing to the bulk chitosan solution and external saturation of the prefabricated chitosan particles with poly(IC) by incubation of the empty particles in the poly(IC) solution.

1. External Saturation with poly(IC)

Particles prefabricated using Modified Electrospray methods, normally 20 to 50 mg dry total, were placed in 0.6 ml of poly(IC) (VWR International, Cat. #IC10270810) solubilized in PBS, 3.0 mg/ml. After 2 hours of gentle shaking at room temperature, the particles were centrifuged 5 times for 2 minutes all at 1000 G, each time the supernatant being discarded and replaced with 1.5 ml of distilled water. The resulting suspension of the washed particles was freeze-dried overnight.

2. Measurement of poly(IC) in Solution Using Ethidium Homodimer.

To determine the concentration of poly(IC) in solution, the effect of the 20-25-fold fluorescence enhancements upon intercalation of Ethidium derivatives was used.

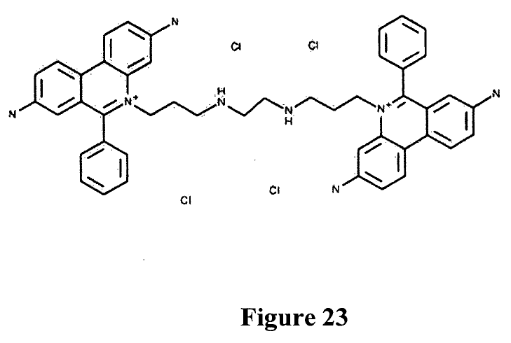

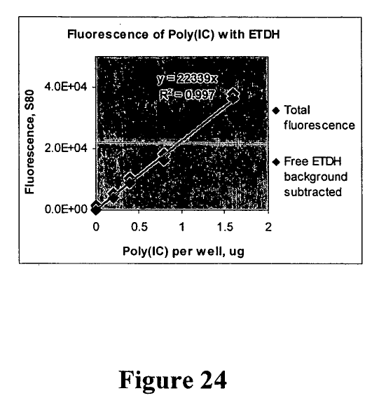

Ethidium Homodimer (ETDH; Sigma-Aldrich, Cat. #46043) is known to form specific complexes with DNA, RNA and even with free nucleotides, due to its chelate structure (FIG. 23). Consequently, it was considered the most suitable fluorescent intercalating agent for measuring poly(IC). Bio-Tek KC-4 multifunctional plate reader was used to measure poly(IC) intercalated with Ethidium Homodimer (ETDH) in standard clear 96-well plates (FIG. 24). Conditions for conduct measurement are shown in table 1.

TABLE 1

| Buffer | PBS |
|---|---|
| Total volume per well | 120 μl |
| Total ETDH | 0.4 μg |
| Poly(IC), max | 2 μg |
| Excitation wave | 535 nm |
| Emission wave | 645 nm |
| Sensitivity | 70-100 |

3. Measuring poly(IC) in the Particles.

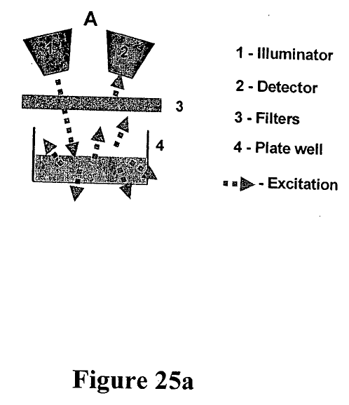

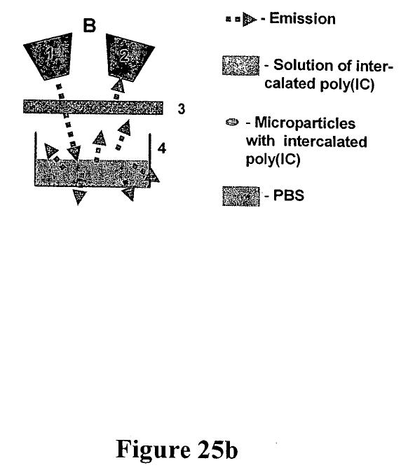

It was found possible to carry out semi-quantitative estimation of poly(IC) contents in the solid chitosan particles using KC-4 reader. Microparticles in watery solutions could be regarded as sufficiently transparent and randomly scattering objects, thanks to their small sizes. Therefore, upon intercalation of ETDH in the surface-bound molecules of poly(IC), and further diffusion inside the particles containing the rest of poly(IC), significant part of the ETDH fluorescence can be collected by the KC-4 reader (FIG. 25).

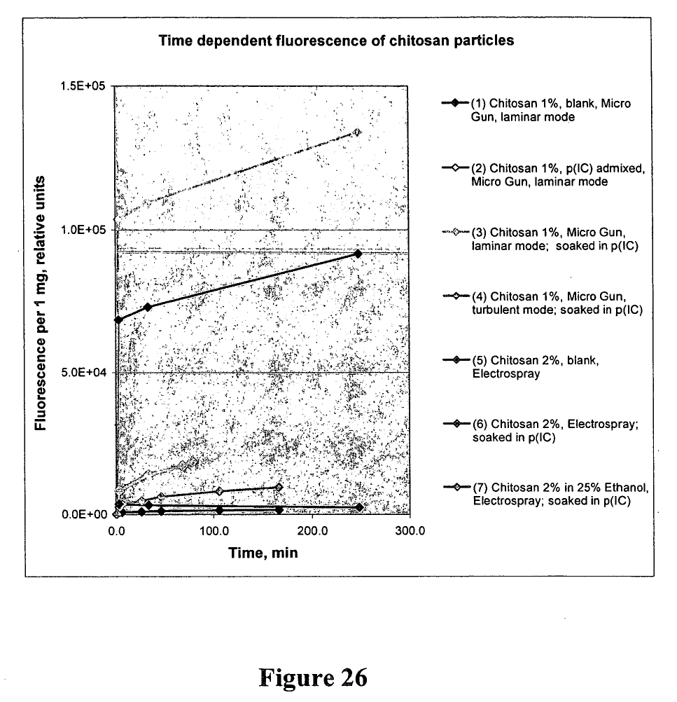

External saturation of the particles with poly(IC) by soaking them in solution has been found much superior than direct admixing poly(IC) in the chitosan solution, which is demonstrated in FIG. 26. Time dependent fluorescence of the poly(IC) particles in the presence of ETDH has demonstrated two distinct phases: immediate intercalation of the easily accessible surface poly(IC) molecules accompanied by fast (a few seconds) buildup of fluorescence, and steady increase of the fluorescence due to slow penetration of ETDH deep in the particles. It has been considered necessary to obtain particles with maximal surface loading, i.e. demonstrating enhanced fast buildup of fluorescence.

The following tentative order of efficiency has been found for the protocols of preparation of the chitosan/poly(IC) particles:

| | | | | | | | |
|---|---|---|---|---|---|---|---|
| Micro | | Micro | | Micro | | Electro- | |
| Gun | | Gun | | Gun | | spray; | |
| Laminar | < | Laminar | < | Plume | < | chitosan in | < |
| mode; | | mode; | | mode; | | water; | |
| p(IC) | | p(IC) | | p(IC) | | p(IC) | |
| admixing | | soaking | | soaking | | soaking | |

| | |
|---|---|
| | Electrospray; |
| < | chitosan in 25% |
| | ethanol; |
| | p(IC) soaking |

The easily accessible surface molecules of poly(IC) in the best particles prepared using Electrospray has comprised 4.7 μg poly(IC) per 1 mg of particles, which was ˜12 times higher than for the particles prepared using Micro Gun by direct admixing (graphs 7 and 2 in FIG. 26, respectively).

4. Low Release of poly(IC) from Chitosan Particles.

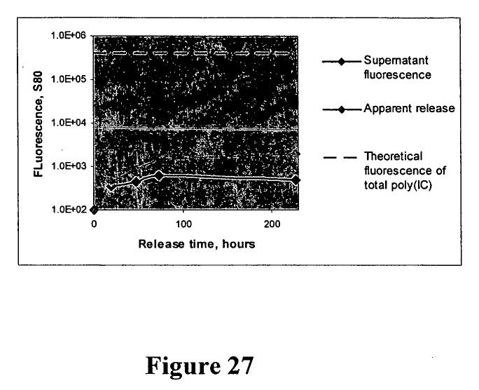

Particles prepared by direct admixing of poly(IC) to chitosan solution, 10 mg dry weight were placed in 1 ml of PBS in a plastic test tube, sealed and incubated on shaker at 37° C. for 9 days. The particles were centrifuged at certain moments of time at 1000 G for 5 minutes; the supernatant was taken for the fluorescence assay in the presence of ETDH as described above and replaced for the fresh PBS. The observed release has occurred insignificant, less than 0.5% of theoretical maximum over 227 hours (FIG. 27). Meager release of poly(IC) from chitosan particles has been found for the particles obtained in both Admixing and Saturation methods of fabrication. In the case when particles are to be phagocytosed this occurrence can be not of much importance.

### EXAMPLE 9

### Multifunctional Chitosan Particles Loaded with OVA and poly(IC)

Ovalbumin (OVA) is a 45 kDa glycoprotein that can be used as a model antigen in immunological experiments. Two methods of preparation of the OVA/poly(IC) loaded chitosan particles were used: Admixing to the bulk chitosan solution and external saturation of the microparticles with OVA/poly(IC).

1. Chitosan Microparticles Prepared by External Saturation with OVA Alone or with a Combination of OVA with poly(IC).

Particles prefabricated by Electrospray, 20 to 50 mg dry weight total were placed in 1.5 ml of 30 mg/ml OVA (Sigma-Aldrich, Cat. #A-5503), or 30 mg/ml OVA_{—}2 mg/ml poly(IC) for 2 hours on a rocker at room temperature. After 2 hours of gentle shaking the particles were centrifuged 5 times at 1000 G, each time the supernatant was discarded and replaced with 1.5 ml of distilled water. The resulting suspension of thus washed particles was freeze-dried overnight.

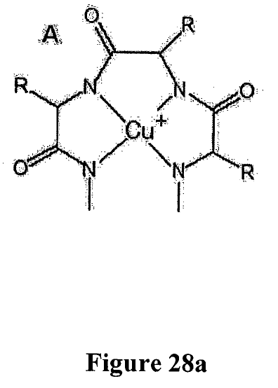

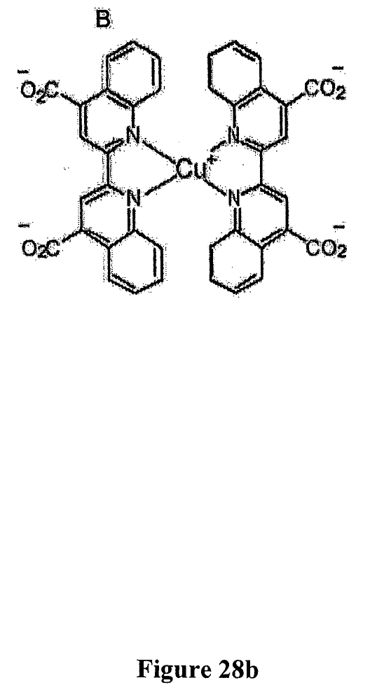

2. Bicinchoninic Acid Assay of OVA Bicinchoninic Acid (BCA) Assay of Proteins is Based on Two Main Steps:
The first step is a Biuret reaction which reduced Cu⁺² to Cu⁺¹;
In the second step Bicinchoninic Acid (BCA) substitutes peptide groups in the Biuret complex to form a bis-chelate complex with Cu⁺¹ which is purple colored and detectable at 562 nm (FIG. 28).

Commercially available BCA kits (e.g. Sigma-Aldrich, Cat. # BCA1) usually contain BCA, Tartrate/Bicarbonate buffer (pH 11.25), and 4% copper sulfate solution. Immediately before the assay, 50 parts of standard alkaline BCA solution are mixed with 1 part of 4% copper sulfate solution to be used as the assay system.

Proteins can be measured in the BCA assay both in solution and in insoluble objects (microparticles suspended in buffer; samples of insoluble protein-containing films; etc.). Heterophase systems, however, require longer incubation of the samples in the BCA solution and at higher temperature (60° C. towards 37° C. for proteins in solutions).

3. Assays of OVA in Solutions and in Insoluble Objects

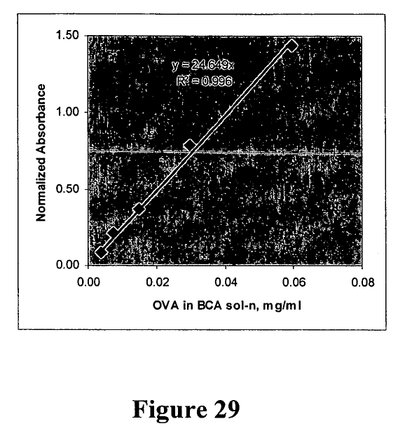

Fresh BCA assay solution was calibrated by OVA standards in order to determine the area of linear response which occurred stretching up to 60 μg of protein per ml of the assay solution (FIG. 29).

OVA in solution has been measured as follows:

Aliquots of protein solutions were added to the necessary excesses of the assay solution to guarantee the final optical extinction no more than 2, and a linear response of the assay altogether. The analytes were incubated for 1 hour in a rocker at 37° C. All readings were corrected to the reading of the bank sample containing zero protein.

OVA in microparticles has been measured as follows:

Samples of dry microparticles (about 1 mg each) were weighed on the analytical scaled (Mettler-Toledo XS105) with 0.01 mg accuracy and suspended in a corresponding excess of the BCA assay solution precalculated as to provide linear response and acceptable optical density (<2 o.u.). The samples sealed in the test tubes were incubated either in the rocker for 4 hours at 37° C., or in water bath for 1 hour at 60° C. with occasional tumbling. In both cases, the incubation times were determined experimentally to provide complete reducing of divalent copper to monovalent copper by molecules of the protein. The tubes were centrifuged at 500 g for 5 minutes. to separate particles, and clear colored solutions were read on a spectrophotometer at 562 nm. All readings were corrected to the reading of the sample obtained with blank particles containing no protein.

### EXAMPLE 10

### Multifunctional Microparticles with Moderate Loading Proteins and Nucleic Acids

1. Estimation of the OVA Contents in Microparticles.

When the protein is added to the particles via direct admixing, it was found that different methods of particle preparation seem to have had no significant effect on the final contents of the OVA. However, the result was different when the protein was loaded by external saturation of prefabricated particles (soaking). External saturation created microparticles with 3-5 times higher final OVA concentrations.

Modified Electrospray and Micro Spray Gun allow for prefabrication of small particles with very high surface areas that exhibit the geometry and shape of “crumpled paper”. The small sizes of the particles and large surface areas microparticles contributed to the high absorption capacity, rather than to release rate.

The high surface areas provide the large external surfaces for the NA and proteins to attach via external saturation of the microparticles in the solutions.

TABLE 2
Final contents of OVA in chitosan microparticles prepared
by different methods

| | | | Relative OVA, by |
|---|---|---|---|
| | Method of | | BCA assay, % |
| Initial system | Preparation | Loading of OVA | w/w |
| 1.5% Chitosan; | Electrospray | Direct admixing | 11.64 |
| 3% OVA | | | |
| 1.5% Chitosan; | Electrospray | Soaking in OVA, | 73.6 |
| 25% Ethanol | | 30 mg/ml for 3 | |
| | | hours | |
| 1.5% Chitosan; | Electrospray | Soaking in OVA, | 23.7 |
| 25% Ethanol | | 30 mg/ml/ + | |
| | | poly(IC), 2 | |
| | | mg/ml, for 3 | |
| | | hours | |

2. Measuring Time Release of Ovalbumin from Microparticles

Samples of loaded particles, 10.0 mg dry weight each were placed in 1.0 ml of PBS in plastic test tubes, sealed with Parafilm and incubated on shaker at 37° C. for up to 18 days. The tubes were centrifuged at certain moments of time at 1000 G for 5 minutes; the supernatant was taken for the BCA assay as described before.

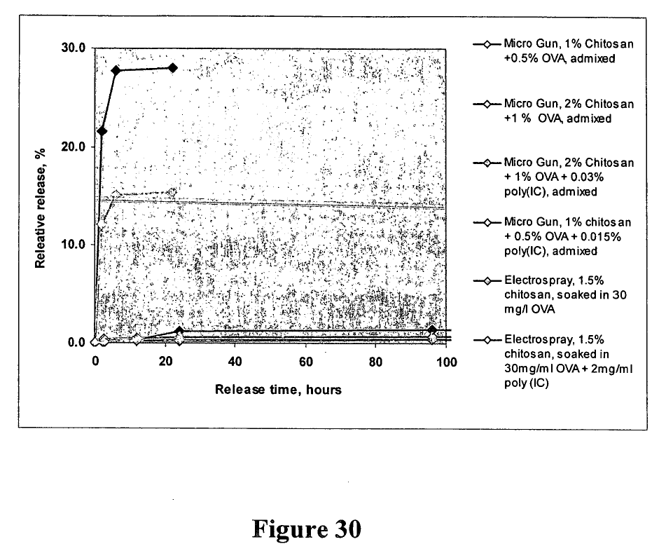

Colossal differences have been found in the release profile of OVA from the particles prepared by admixing protein, and by soaking prefabricated particles in the protein solution (FIG. 30). For the former, the altogether release never exceeded 2% of the total load in many days; for the latter, the release achieved 15% -30% and took place within the first 7-10 hours. Alike to the effect on the total contents of OVA, simultaneous saturation of the particles with OVA and poly(IC) seemingly decreased the release of OVA (FIG. 30).

### EXAMPLE 11

### Chitosan Particles Highly Loaded with polyinosinic-polycytidylic Acid

Accessible surface-attached molecules of poly(IC) in the best particles prepared using Electrospray contained 4.7 μg poly(IC) per 1 mg of particles. The sizes of the particles occurred distributed from ˜5 to ˜50 microns, and tripolyphosphate (TPP) was used as crosslinker. The nearest goal was therefore set to increase sorption capacity of the particles.

To enhance sorption capacity of the particles, it was found desirable to change a chitosan crosslinker. Sodium sulfate Na₂SO₄ (10% in distilled water if not specified otherwise) has been chosen as prospective gelation crosslinker creating softer particles, and in the same time being much weaker competitor towards binding phosphate groups of nucleic acids [Berthold, et al., J Controlled Release, 39:17-25 (1996)].

Supra micro (i.e. big) - and submicron (small) Protasan particles loaded with poly(IC) were prepared. Supra micro particles (20 to 700 microns) deemed to be used as chemokine or drug carriers, or to activate extracellular TLR-3 immunity pathway; they should avoid being engulfed by cells.

On the other hand, immunization is known to be effective when nucleic acids and antigens are carried by smaller particles (0.5 to 10 um) that can be engulfed by antigen presenting cells and processed via internal FADD/RIP/TRAF-2 pathway which is central for the activation of primary innate immune response.

### EXAMPLE 12

### Supra-Micron Protasan Particles Highly Loaded with polyinosinic-polycvtidylic Acid

Bigger particles (100-200 micron) were obtained spraying 10 ml of 2% solution of PROTASAN UP CL 213 (NovaMatrix, Norway, cat # 420101) in 1% acetic acid using Micro Air Gun, in a laminar mode over a receiving pan containing 100 ml Na₂SO₄ solution pH 5.5.

Protasan was manufactured and documented in accordance with US FDA guidelines for cGMP (21 CFR 210, 211).

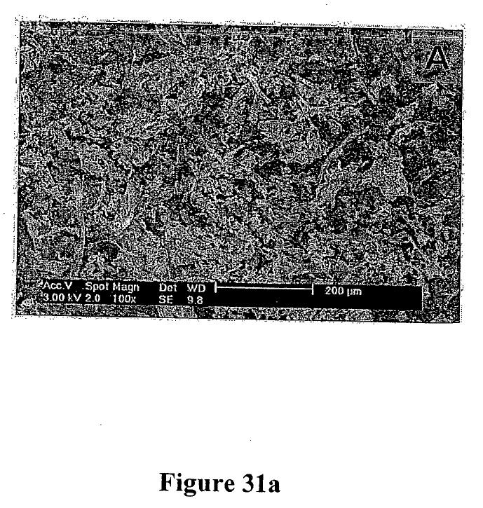

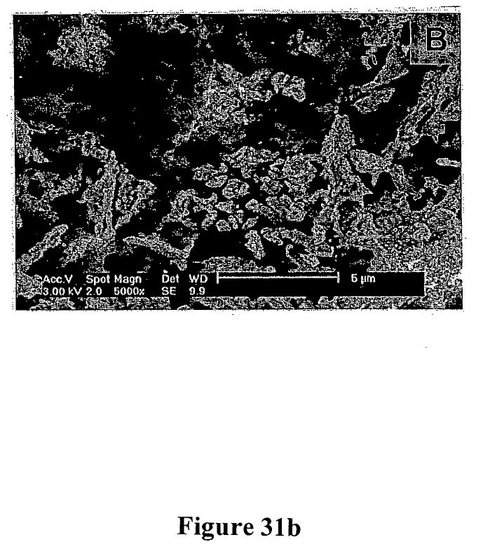

The particles were washed in distilled water 6 times using recursive centrifugation/resuspension procedure and freeze dried overnight. The resulting particles have occurred irregular and spongy fragments from ˜10 to 200 microns (FIG. 31A).

1. Poly(IC): Solubilization and Measurement

50 mg of poly(IC) (Amersham, cat. # 27-4729-01) was dissolved in 35 ml of 1 % NaCl overnight, as recommended by the manufacturer. The final solution had optical absorbance at 260 nm A₂₆₀ ˜14.0 which corresponded to 700 μg of pure double stranded poly(IC) per ml. Therefore, the total contents of poly(IC) in the Amersham preparation was about 49-50%, all other components being buffering salts.

The amount of poly(IC) in the particles was calculated as the difference between the poly(IC) added to the system, and poly(IC) remaining in the aqueous phase after particle precipitation [Bivas-Benitz, et al., Int. J. Pharm., 266:17-27 (2003)]. To measure concentration of poly(IC) in solution, direct reading of poly(IC) UV spectra has been used instead of fluorescence methods, due to very high concentration of poly(IC) involved in the preparations.

2. Measuring Sorption of poly(IC) by Prefabricated Particles

Particles of a known weight were suspended in a volume from 0.5 to 5 ml of acetate or phosphate buffer containing from 50 to 700 ug/ml poly(IC) in different experiments. The suspensions were vortexed intensively for 5 minutes, and then kept vortexed at intermediate level or rocked for another 15 minutes. Afterwards, the suspensions were centrifuged at 7000 g for 5 minutes, and the concentration of poly(IC) in the supernatant was measured in spectrophotometer using a 1-cm quartz cell. The concentration of poly(IC) was determined using the difference of the optical absorptions at 260 and 400 nm, where every 1 optical unit corresponded to ˜50 μμg/ml poly(IC) (American Biosciences, specification for the Product #27-4732).

3. Sorption Capacity of the Supra Micron Protasan Particles as it Depends on pH

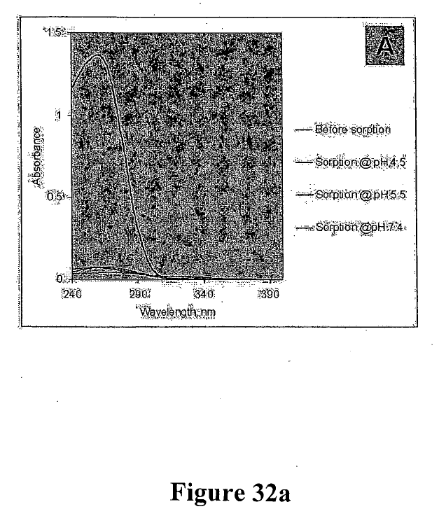

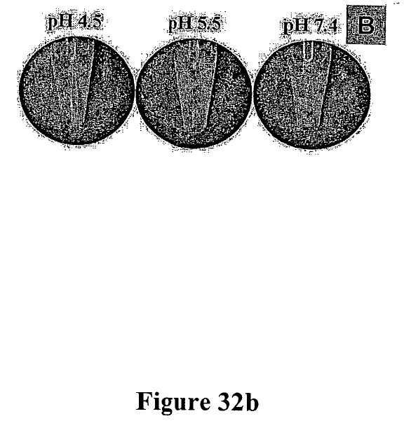

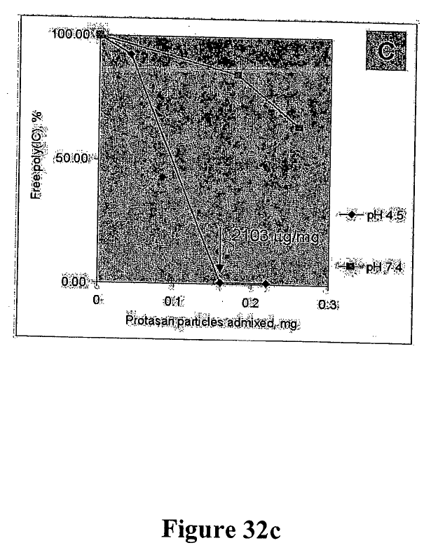

Solution of poly(IC) 0.7 mg/ml was mixed 1:1 with three different buffers: 1% acetate buffer pH 4.5; 1% acetate buffer pH 5.5; PBS pH 7.4. Dry Protasan particles, 1.0+−0.04 mg in each portion were suspended in 1.0 ml volumes of (poly(IC) : buffer) mixed solutions. After vortexing and centrifugation, the unabsorbed rest of poly(IC) was measured as described. All three samples have shown similar sorption capacity of the Protasan particles, equal to or exceeding 0.7 mg poly(IC) per mg dry empty particles (FIG. 32a). The physical states of the samples were different though. The sample at pH 4.5 has shown the most compact precipitation of the particles, whereas the sample at pH 5.5 demonstrated an ample and incompressible pellet, and the sample at pH 7.4 has shown intermediate compressing (FIG. 32b). Maximal sorption capacity of the particles was later found highest at pH 4.5 (FIG. 32c).

As a result of the above findings, all subsequent experiments on sorption of poly(IC) by various particles have been conducted at pH 4.5

Experiments on sorption of poly(IC) on Protasan particles prefabricated using sodium sulfate as crosslinker demonstrated that maximal sorption capacity at pH 4.5 exceeded 2 mg poly(IC) per 1 mg empty particles. This result have shown ˜400 improvement towards results with TPP crosslinker.

### EXAMPLE 13

### Submicron Protasan Particles Highly Loaded with poly(IC)

Submicron particles were fabricated using slow precipitation of the Protasan/Poly(IC)/Crosslinker agglomerates from diluted solutions.

200 ml of poly(IC) solution, 200 μg/ml in 0.1% acetate buffer pH 4.5 was being added by drops within 15 minutes to 200 ml of Protasan solution 200 μg/ml in 0.1% acetate buffer at constant stirring at room temperature. The resulting solution was stirred for 1 hour at 30° C., afterwards 400 ml of 10% solution of sodium sulfate was added by drops within 15 minutes. The final 800 ml of the combined solution was stirred for 2 hours at 30° C., and then precipitated by centrifuging at 5000G. The pellet was washed twice in distilled water as described above, resuspended in water, then filtered through 40 um BD Falcon cell strainer (BD Biosciences, cat.# 352340), then precipitated again, and finally resuspended in water at ˜5 mg/ml. The sizes of these particles were found in the range of 1-20 microns (FIG. 31B). Sorption capacity of these particles appeared to be 1 mg/mg.

### EXAMPLE 14

### Hydrophobic Cationic PLGA/PEI/POLY(IC) Combined Particles

To fabricate PLGA/PEI /poly(IC) particles, various modification of the protocol of Bivas-Benita et al. has been used [Bivas-Benita, et al., Eur. Jour. of Pharmaceutics and Biopharmaceutics, 58:1-6 (2004)]. In effect, solutions of PLGA in dichloromethane and PEI in acetone were combined in different proportions, and microparticles were obtained using sonic emulsification, Air gun and Electrospray atomization.

1. Sonic Emulsification

500 mg PLGA was dissolved in 5 ml dichloromethane and combined with 100 mg PEI dissolved in 5 ml acetone (5:1 final PLGA:PEI ratio). The combined solution was poured dropwise in 50 ml 10% NaCi water solution kept under constant sonication in Branson-1510 sonication bath at room temperature. Sodium chloride has been introduced to facilitate dispersing the organic phase and resuspending during the washing procedure. The mixture was being sonicated for another 4 hours at elevated temperature (50° C.) to eliminate the volatile solvents. The resulting PLGA/PEI particles were washed /sedimented 4 times, as described before, and freeze dried. It was found possible to reduce the number of washing passes due to elimination of persistent surfactants.

2. Air Gun and Electrospray of PLGA/PEI Solutions over NaCl Receiving Water Solution

The above described 5:1 PLGA: PEI solution in CH₂Cl₂/acetone was sprayed over 10% NaCl using Micro Air Gun and Electrospray over turbulent 10% NaCl solution. The collected microparticles were washed/sedimented 4 times and freeze dried.

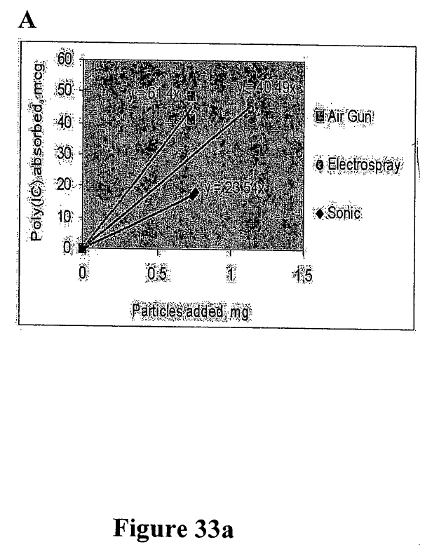

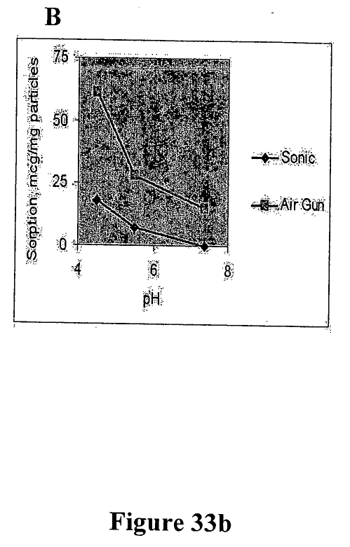

Sorption of poly(IC) by the particles obtained without surfactants has been tested as described above in the poly(IC) solution ˜70 μg/ml. The general sorption capacity was found improved to about an order to compare with emulsion technique; the pH 4.5 acetate buffer has been found again the most suitable for the sorption (FIGS. 33a and b).

Whereas sorption capacity of the particles obtained using the Air Micro Gun has occurred somewhat higher than for the particles from Electrospray, the altogether shapes and size distribution was better in the latter case. Air Gun actually produced irregular agglomerates of the size higher than 10 microns (not shown), and the particles form Electrospray were spheroids of the size range 3-10 microns (data not shown).

3. Particles Obtained Using Electrospray over Dry Metallic Electrode

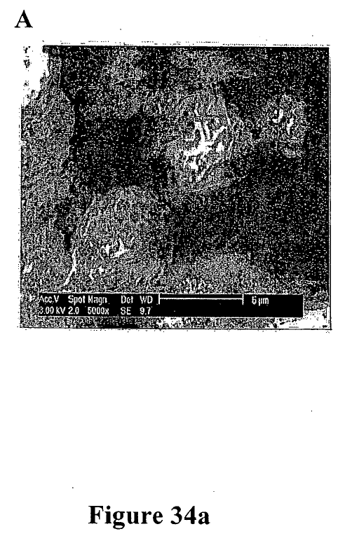

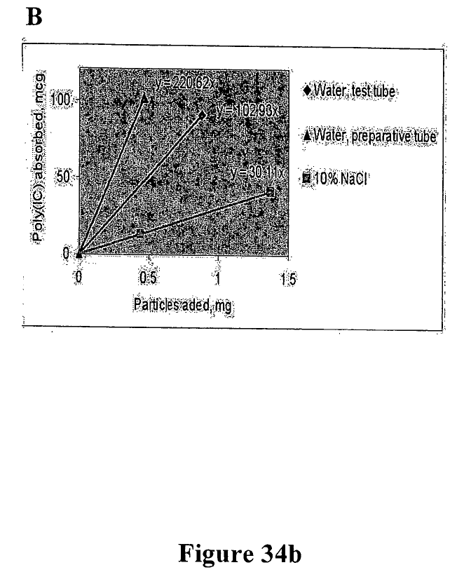

It was found possible to receive the electrodispersed particles onto dry metallic electrode (stainless steel pan) and solubilize them afterwards. In order to further rise sorption capacity of the particles, the PLGA:PEI ratio was increased to 2:1, i.e. 500 mg PLGA versus 250 mg PEI in the same volumes of CH₂Cl₂ and acetone, as before. The particles collected onto dry electrode looked as 3-7 micron spheroids (FIG. 34a).

It is safe to conclude that fabrication PLGA/PEI particles with Electrospray over dry electrode with subsequent solubilization of the deposit in distilled water looks by far superior towards various emulsion methods or dispersion over watery solutions.

4. Fluorescent PLGA/PEI/FITC Particles for Observations of Phagocytosis

To facilitate initiation of experiments on phagocytosis of poly(IC)-carrying particles, a simplified version of fluorescent labeling has been introduced. The particles were synthesized according the above described protocol for Electrospray over dry electrode, with 2 mg FITC (Sigma-Aldrich, cat. # F-7250) in 1 ml of 95% ethanol added to a standard combined PLGA:PEI=2:1 solution.

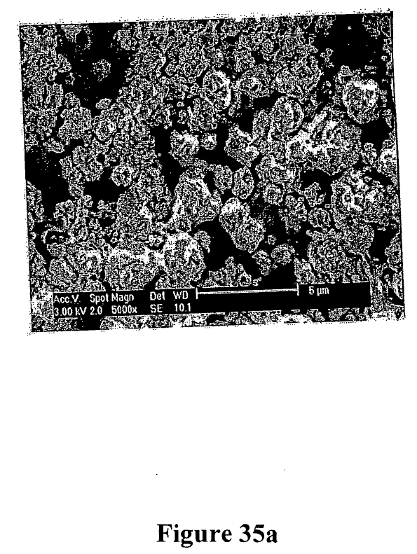

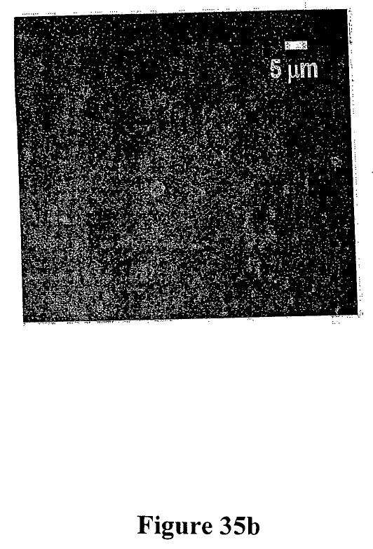

The particles were resuspended in distilled water with sonication and washed 4 times out of the free FITC. The fourth wash has shown zero traces of free FITC. The resultant pellet of intensive yellow color was freeze dried overnight and charged with poly(IC) using standard protocol described above. The sorption capacity was found lower than for the particles without FITC obtained earlier:˜70 μg/mg towards 100-200 μg/mg. The particles were irregular and somewhat spongy spheroids of 0.5-5 μm size, showing bright green fluorescence (FIG. 35)

5. Preliminary Results on Induction of Interferon in Human Dendritic cells.

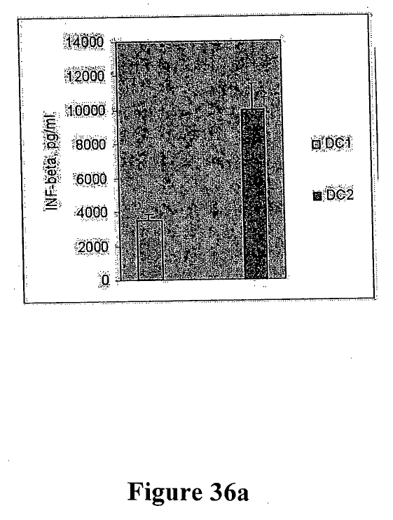

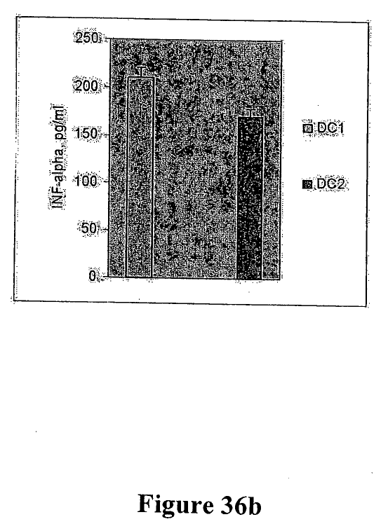

About 50,000 primary freshly sorted DC1 or DC2 subset human cells were treated with PLGA-PEI-poly(IC) particles, and culture supernatants were collected 24 hours later and subjected to ELISA for human IFN-α and β. Statistically consistent levels of both Beta and Alpha interferon were found for both subsets (FIG. 36).

TABLE 3
Cumulative table of the produced particles

| | | | | Sorption of |
|---|---|---|---|---|
| | | | | poly(IC), μg/mg |
| Materials | Method of synthesis | Size, μm | Shape | particles |
| Protasan | Air Gun over | 10-200 | Irregular | >2000 |
| | 10% Na₂SO₄ | | fragments | |
| Protasan | Precipitation at | 1-20 | Irregular | >1000 |
| | 5% Na₂SO₄ | | fragments | |
| PLGA/P | Classical | 0.3-3 | Spheres | 3.7 |
| EI 10:1 | emulsification | | | |
| PLGA/P | Zero surfactants, | 3-10 | Spheroids | 23.5 |
| EI 5:1 | sonication in | | | |
| | 10% NaCl | | | |
| PLGA/P | Air Gun over | >10 | Agglomerates | 61.4 |
| EI 5:1 | 10% NaCl | | | |
| PLGA/P | Electrospray over | 3-7 | Spheroids | 40.5 |
| EI 5:1 | 10% NaCl | | | |
| PLGA/P | Electrospray over | 3-7 | Spheroids | 30.1 |
| EI 2:1 | dry electrode, | | | |
| | solubilization in | | | |
| | 10% NaCl | | | |
| PLGA/P | Electrospray over | 3-7 | Spheroids | 102.9; 220.6 |
| EI 2:1 | dry electrode, | | | |
| | solubilization in | | | |
| | water | | | |
| PCL/PEI | Electrospray over | 3-5 | Spheroids | 378.1 |
| 2:1 | dry electrode, | | | |
| | solubilization in | | | |
| | water | | | |
| PLGA/P | Electrospray over | 1-3 | Spheroids | ˜70 |
| EI/FITC | dry electrode, | | | |
| | solubilization in | | | |
| | water | | | |

### EXAMPLE 15

### Cross-Signaling Defense Pathways Against Non-Viral Pathogens

The cross-signaling strategies may be useful in combating bacterial as well as virus-related disease. To evaluate these possibilities, and to confirm that microparticles carrying stimulators of the TLR/FADD-pathway exert potent adjuvant properties including cross-priming of antigen-specific T-cells, OT-1 transgenic mouse that expresses the T cell receptor (TCR) for chicken ovalbumin (OVA) will be used as a model. A majority of CD8 T cells in these animals express a single Vα2+Vβ5+TCR that recognizes an ovalbumin peptide (SIINFEKL) in association with a K^{b} molecule. These particles can be modified to express the OVA gene or can be directly loaded with the protein. Purified CD8⁺ Vα2 cells labeled with CFSE are adoptively transferred into the animals. After three days, the OVA containing particles (gene or protein) are inoculated into animals (i.p.). This method was recently shown to demonstrate increased cross-presentation of OVA from gp96 expressing cells, to OVA-specific T-cells. By using this approach, microencapsulation strategies that involve stimulation of the innate immune response can be shown to be efficient modulators of the adaptive immune response.

### EXAMPLE 16

### Demonstration that PLGA/PEI or Protasan Microparticles Loaded with poly (IC) Induces INF β Production in 293 Cells by Activating the Extracellular TLR3 Pathway

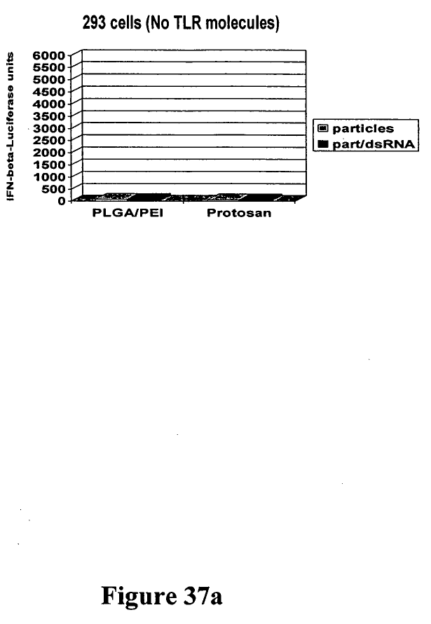

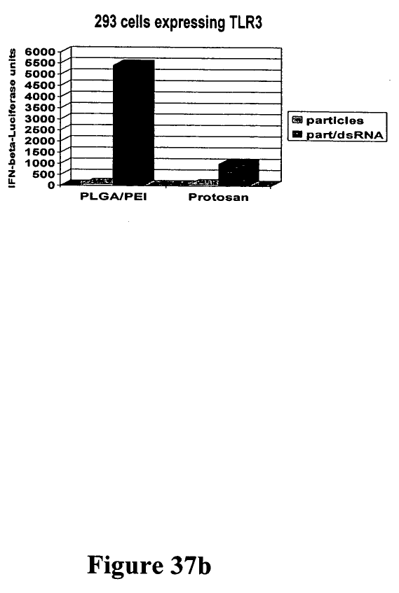

293 cells expressing or not expressing TLR3, a receptor for exogenous dsRNA, were transfected with a luciferase gene under control of the IFN-beta promoter and exposed to PLGA/PEI particles (with and without amalgamated dsRNA) or Protosan particles (with and without amalgamated dsRNA). The exposure time to the particles was between 3-6 hours. As shown in FIG. 37b, only particles with dsRNA were able to trigger extracellular TLR3 mediated activation of the luciferase gene. As controls, 293 cells without the TLR3 receptor were transfected with a luciferase gene under control of the IFN-beta promoter and exposed to PLGA/PEI particles (with and without amalgamated dsRNA) or Protosan particles (with and without amalgamated dsRNA (FIG. 37a). No significant luciferase activity was detected, indicating that only microparticles with dsRNA were able to activate the IFN-beta pathway via TLR3. The 293 cells have a very weak intracellular pathway, thus, the reason to largely activate the TLR3 pathway (data not shown).

Control: As a further control exogenous dsRNA was added to the 293 cells expressing or not TLR3. Both types of cells were transfected with a luciferase gene under control of the IFN-beta promoter and treated with exogenous dsRNA. Only cells expressing TLR3 were able to be activated by dsRNA, to transcriptionally activate the IFN beta promoter.

### EXAMPLE 17

### Demonstration that PLGA/PEI or Protasan Microparticles Loaded with poly (IC) Induces INFα Production in DC2 Subset Cells by most Likely Activating the Intracellular Innateosome Pathway

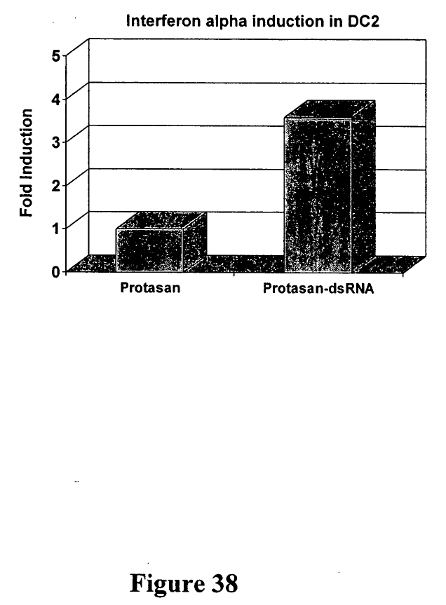

DC2 subsets in peripheral human blood samples were exposed to PLGA/PEI or Protosan particles (with or without amalgamated dsRNA) and monitored for Interferon alpha expression after 3-6 hours of exposure to the particles as shown in FIG. 38. DC2 (plasmacytoid DCs lack TLR 3 and so IFN alpha induction is being triggered by alternate dsRNA signaling pathways), most likely utilizing the intracellular pathway via the “innateosome.”

The preferred embodiments of the compounds and methods of the present invention are intended to be illustrative and not limiting. Modifications and variations can be made by persons skilled in the art in light of the above teachings. It is also conceivable to one skilled in the art that the present invention can be used for other purposes of measuring the acetone level in a gas sample, e.g. for monitoring air quality. Therefore, it should be understood that changes may be made in the particular embodiments disclosed which are within the scope of what is described as defined by the appended claims.

1. A composition for modulating innate immune system in a mammal, said composition comprising
a microparticle comprising a polycationic polymer;
a modulator of FADD-dependent pathway; and
a modulator of TLR pathway,
wherein said modulator of FADD-dependent pathway and said modulator of TLR pathway are associated with said microparticle, and wherein said microparticle is capable of being phagocytosed by an antigen presenting cell.

2. The composition of claim 1, wherein said modulator of FADD-dependent pathway is selected from the group consisting of dsRNA, poly(IC), a component of the FADD-dependent pathway, a DNA plasmid encoding a component of the FADD-dependent pathway, a bacterium, and a fungus.

3. The composition of claim 2, wherein the FADD-dependent pathway modulator is a dsRNA encoding FADD.

4. The composition of claim 2, wherein the FADD-dependent pathway modulator is a dsRNA representing a silencing RNAi capable of suppressing the FADD-dependent pathway.

5. The composition of claim 4, wherein the silencing RNAi suppresses FADD expression.

6. The composition of claim 1, wherein said modulator of TLR pathway is selected from the group consisting of dsRNA, poly (IC), a synthetic mimetic of viral dsRNA, and a ligand for TLR, a bacterium, and a fungus.

7. The composition of claim 1, wherein said modulator of FADD-dependent pathway and modulator of TLR-dependent pathway are the same dsRNA molecule.

8. The composition of claim 1, wherein said microparticle is further coated with a targeting molecule that binds specifically to an antigen presenting cell.

9. The composition of claim 8, wherein said targeting molecule is an antibody.

10. The composition of claim 9, wherein said targeting molecule is heat shock protein gp96.

11. The composition of claim 1, further comprising a poly(lactide-co-glycolide)(PLGA) matrix containing a cytokine or an antigen, wherein said microparticle is encapsulated in said matrix.

12. The composition of claim 1, further comprising a cytokine encapsulated in said microparticle.

13. The composition of claim 12, wherein said cytokine is selected from the group consisting of IL-12, IL-1α, IL-1β, IL-15, IL-18, IFNα, IFNβ, IFNγ, IL-4, IL-10, IL-6, IL-17, IL-16, TNFα, and MIF.

14. The composition of claim 13, wherein said microparticle further comprising one or more hydrophobic polymers so that a desired release rate of cytokine is achieved.

15. The composition of claim 14, wherein said one or more hydrophobic polymers comprise PLGA, poly(caprolactone) or poly(oxybutirate).

16. The composition of claim 13, wherein said microparticle ftirther comprising an amphiphilic polymer.

17. The composition of claim 16, wherein said amphiphilic polymer is poly(ethylene imine) (PEI).

18. The composition of claim 1, wherein said composition further comprising a tumor antigen or a DNA encoding a tumor antigen, and wherein said tumor antigen or DNA encoding a tumor antigen is associated with said microparticle.

19. The composition of claim 1, wherein said microparticle has a diameter in the range of about 0.5 μm to about 20 μm.

20. The composition of claim 1, wherein said polycationic polymer is chitosan.

21. The composition of claim 1, further comprising a pharmaceutically acceptable carrier.

22. A composition for modulating immune system in a mammal, comprising phagocytosable chitosan microparticles loaded with a nucleic acid and a protein.

23. The composition of claim 22, wherein said nucleic acid is a dsRNA, poly (IC), a synthetic mimetic of viral dsRNA, or DNA molecule.

24. The composition of claim 22, wherein said protein is a cytokine.

25. The composition of claim 24, wherein said cytokine is selected from the group consisting of IL-12, IL-1α, IL-1β, IL-15, IL-18, IFNα, IFNβ, IFNγ, IL-4, IL-10, IL-6, IL-17, IL-16, TNFα, and MIF.

26. The composition of claim 22, wherein said protein is an antibody that binds an antigen presenting cell.

27. The composition of claim 22, wherein said nucleic acid is a dsRNA and said protein is a TLR ligand.

28. The composition of claim 22, wherein said nucleic acid is a dsRNA and said protein is FADD.

29. The composition of claim 22, wherein said chitosan particle further comprises a hydrophobic polymer.

30. The composition of claim 29, wherein said hydrophobic polymer is selected from the group consisting of PLGA, poly(caprolactone) and poly(oxybutirate).

31. The composition of claim 22, wherein said chitosan particle further comprises PEI.

32. The composition of claim 22, further comprising a pharmaceutically acceptable carrier.

33. A method for treating viral, bacterial or fungal infection in a mammal, comprising administering to said subject an effective amount of the composition of claim 22.

34. The method of claim 33, wherein said viral infection is caused by human immunodeficiency virus (HIV), influenza virus (INV), encephalomyocarditis virus (EMCV), stomatitis virus (VSV), parainfluenza virus, rhinovirus, hepatitis A virus, hepatitis B virus, hepatitis C virus, apthovirus, coxsackievirus, Rubella virus, rotavirus, Denque virus, yellow fever virus, Japanese encephalitis virus, infectious bronchitis virus, Porcine transmissible gastroenteric virus, respiratory syncytial virus, papillomavirus, Herpes simplex virus, varicellovirus, Cytomegalovirus, variolavirus, Vacciniavirus, suipoxvirus or coronavirus.

35. The method of claim 34, wherein said viral infection is caused by HIV, INV, EMCV, or VSV.

36. A method for treating cancer in a mammal, comprising administering to said subject an effective amount of the composition of claim 22.

37. The method of claim 36, wherein said cancer is breast cancer, colon-rectal cancer, lung cancer, prostate cancer, skin cancer, osteocarcinoma, or liver cancer.

38. A composition for modulating immune response in a mammal, said composition comprising:
a microparticle comprising a polycationic polymer;
a dsRNA or poly (IC) as an innate immune response booster; and
an antigen,
wherein said dsRNA or poly (IC) and said antigen are associated with said microparticle and wherein said microparticle is capable of being phagocytosed by an antigen presenting cell.

39. The composition of claim 38, further comprising a cytokine, wherein said cytokine is associated with said microparticle.

40. The composition of claim 39, wherein said cytokine is selected from the group consisting of IL-12, IL-1α, IL-1β, IL-15, IL-18, IFNα, IFNβ, IFNγ, IL-4, IL-10, IL-6, IL-17, IL-16, TNFα, and MIF.

41. The composition of claim 38, further comprising a heatshock protein, wherein said heatshock protein is associated with said microparticle.

42. The composition of claim 38, wherein said dsRNA or poly (IC) and said antigen are associated with said microparticle through surface attachment, encapsulation, or a combination of surface attachment and encapsulation.

43. The composition of claim 38, wherein said immune response is innate immune response.

44. The composition of claim 38, wherein said immune response is adaptive immune response.

45. A composition for modulating innate immune response in a mammal, said composition comprising:
a microparticle comprising a polycationic polymer;
an immune activator capable of inducing the formation of an innateosome complex regulating TBK-1/IKK-δ-mediated activation of IRF3, and
a modulator of TLR pathway,
wherein said activator for an innateosome complex and said modulator of TLR pathway are associated with said microparticle and wherein said microparticle is capable of being phagocytosed by an antigen presenting cell.

46. The composition of 45, wherein said immune activator is a dsRNA.

47. The composition of 46, wherein said dsRNA is a viral dsRNA.

48. A method for preparing a multifunctional microparticle for immune modulation of a mammal, comprising:
(a) fabricating chitosan microparticles by precipitation, gelation and spray
(b) incubating the chitosan microparticles in a solution comprising a nucleic acid, a protein, or both.

49. The method of claim 48, following step (b), further comprising the steps of:
(c) washing the chitosan microparticles after incubation; and
(d) drying the washed chitosan microparticles.

50. The method of claim 48, wherein said nuclei acid is selected from the group consisting of dsRNA, poly (IC), synthetic mimetic of viral dsRNA, and DNA, wherein said protein is selected from the group consisting of antibodies, cytokines, TLR ligand, gp96, and tumor antigens.

51. The method of claim 50, wherein said cytokine is selected from the group consisting of IL-12, IL-1α, IL-1β, IL-15, IL-18, IFNα, IFNβ, IFNγ, IL-4, IL-10, IL-6, IL-17, IL-16, TNFα, and MIF.

52. The method of claim 48, further comprising:
admixing chitosan with a nucleic acid, a protein, or both before fabricating the chitosan microparticles by precipitation, gelation, and spray.

53. A method for identifying anti-viral genes relating to FADD signaling pathway, comprising:
treating FADD-deficient cells and corresponding wild-type cells with poly (IC);
isolating RNAs from poly (IC)-treated FADD-deficient cells and poly (IC)-treated wild-type cells;
hybridizing the isolated RNAs to a gene array; and
identifying genes that are differentially expressed in poly (IC)-treated FADD-deficient cells comparing to poly (IC)-treated wild-type cells.
